(19) United States
(12) Patent Application Publication
(10) Pub. No.: US 2019/0060027 A1
(43) Pub. Date: Feb. 28, 2019

(54) IN-VIVO IMAGING DEVICE AND IN-VIVO MONITORING CAMERA SYSTEM

(71) Applicant: SHARP KABUSHIKI KAISHA, Osaka (JP)

(72) Inventors: YAN QIAN, Sakai City (JP); KEI URAKAWA, Sakai City (JP); NARAKAZU SHIMOMURA, Sakai City (JP)

(73) Assignees: SHARP KABUSHIKI KAISHA, Sakai City, Osaka (JP); SHARP KABUSHIKI KAISHA, Sakai City, Osaka (JP)

(21) Appl. No.: 16/116,778

(22) Filed: Aug. 29, 2018

(30) Foreign Application Priority Data
Aug. 31, 2017 (JP) 2017-167930

(51) Int. Cl. A61B 90/00; A61B 1/00; A61B 1/005; A61B 1/05; A61B 1/313

(52) U.S. Cl. CPC A61B 90/361; A61B 1/00114; A61B 1/00105; A61B 1/00128; A61B 1/00045; A61B 1/005; A61B 1/053; A61B 1/3132; A61B 1/00147

(57) ABSTRACT

An in-vivo imaging device includes a camera, a support rod insertable into a tubular instrument, and a gripping portion disposed at an end portion of the support rod for gripping the camera. When the support rod is pulled up outward of a body, the gripping portion is deformed and grips the camera by coming into contact with an in-vivo side end portion of the tubular instrument.

### BACKGROUND

### 1. Field

The present disclosure relates to an in-vivo imaging device and an in-vivo monitoring camera system.

### 2. Description of the Related Art

An operator uses an endoscope including an imaging unit so as to internally capture an image of an abdominal cavity. While observing a lesion area, the operator performs a surgery. As a known technique, the following technique is known. An overhead view is secured by fixing a camera onto an abdominal wall so that not only the lesion area of a medical treatment target but also the whole abdominal cavity are internally observed.

Japanese Patent No. 5886481 (filed on Mar. 16, 2016) and Pamphlet of International Publication No. 2016/203864 (published on Dec. 22, 2016) describe an in-vivo monitoring camera system as follows. In order to improve reliability and usability, an imaging unit is joined to one end of a support tube inside a body, and the other end of the support tube is exposed outward of the body. Alternatively, the other end is connected to a tubular instrument which is partially introduced into the body.

Japanese Patent No. 5669666 (filed on Feb. 12, 2015) discloses a medical system having an abdominal-cavity-installed camera which is a medical instrument indwelling the abdominal cavity, and an introduction/collection dedicated tool which introduces the camera into the body and collects the camera from the inside of the body. The introduction/collection dedicated tool is an insertion/collection unit for introducing the camera into the body and collecting the camera from the inside of the body via a trocar introduced into the body by perforating the abdominal wall toward the inside of the abdominal cavity.

According to a technique described in Japanese Patent No. 5886481 (filed on Mar. 16, 2016), Pamphlet of International Publication No. 2016/203864 (published on Dec. 22, 2016), and Japanese Patent No. 5669666 (filed on Feb. 12, 2015), in a case where the camera is fixed onto the abdominal wall and an image of the whole inner portion of the abdominal cavity is captured in an overhead view, there remains room for improvement in that installation work until the camera is fixed onto the abdominal wall may be simplified and the angle and position of the installed camera may be finely adjusted.

Normally, the operator performs a process to pick up a wire of the camera and pull the wire out of the body while gripping the camera with forceps inside the abdominal cavity. During this process, the operator carries out work for joining the camera and the support tube to each other. According to the technique in Japanese Patent No. 5886481 (filed on Mar. 16, 2016), Pamphlet of International Publication No. 2016/203864 (published on Dec. 22, 2016), and Japanese Patent No. 5669666 (filed on Feb. 12, 2015), smooth alignment is not considered when the camera and a joint portion of the support tube are aligned with each other, and thus, the operator sometimes feels a considerable burden. Even after the above-described installation work is complete and the camera is installed on the abdominal wall, there may be a case where the camera is misaligned.

It is desirable to realize an in-vivo imaging device and an in-vivo monitoring camera system, which can simplify work for installing a camera inside a body and which can finely adjust the angle and position of the camera even after the camera is installed.

### SUMMARY

An in-vivo imaging device according to an aspect of the disclosure includes an imaging unit that internally captures an image of a body cavity, a support member insertable into a tubular instrument to be introduced into a body, and a gripping portion that is disposed at an in-vivo side end portion of the support member and grips the imaging unit. When the support member is pulled up outward of the body, the gripping portion is deformed and grips the imaging unit by coming into contact with the in-vivo side end portion of the tubular instrument.

An in-vivo imaging device according to another aspect of the disclosure includes an imaging unit that internally captures an image of a body cavity, a support member insertable into a tubular instrument to be introduced into a body, and a gripping portion that is disposed at an in-vivo side end portion of the support member and grips the imaging unit. The gripping portion has a sucker portion which suctions the imaging unit.

An in-vivo imaging device according to still another aspect of the disclosure includes an imaging unit that internally captures an image of a body cavity, a support member insertable into a tubular instrument to be introduced into a body, and a gripping portion that is disposed at an in-vivo side end portion of the support member and grips the imaging unit. The support member includes an outer member in a tubular shape, an inner member insertable into the outer member, and a gripping mechanism with which the gripping portion grips the imaging unit in conjunction with an upward-downward movement of the inner member. The gripping mechanism includes a claw member which is supported rotatably with respect to the outer member, and a link member which links the claw member and the inner member with each other and which is supported rotatably with respect to the claw member. The link member is supported rotatably with respect to the inner member.

### BRIEF DESCRIPTION OF THE DRAWINGS

FIG. 1 is a schematic view illustrating a configuration of an in-vivo monitoring camera system according to Embodiment 1 of the disclosure;

FIG. 2 is a perspective view illustrating a configuration of an in-vivo imaging device according to Embodiment 1 of the disclosure;

FIGS. 3A to 3C illustrate a support rod of the in-vivo imaging device according to Embodiment 1 of the disclosure, where FIG. 3A is a schematic view illustrating respective configurations of a trocar and the support rod, FIG. 3B is a side view illustrating the configuration of the support rod when viewed from the side in FIG. 3A, and FIG. 3C is a schematic view illustrating a positional relationship between the support rod and the trocar when the in-vivo imaging device is used;

FIGS. 4A to 4E are schematic views illustrating a method of installing a camera in a body according to Embodiment 1 of the disclosure;

FIGS. 5A to 5E are schematic views illustrating the method of installing a camera in a body after a step in FIG. 4E, where FIG. 5A illustrates a step in FIG. 4E, FIGS. 5B to 5D illustrate a step after the step in FIG. 4E, and FIG. 5E is a sectional view taken along line VE-VE in FIG. 5D;

FIG. 6 is a schematic view illustrating a use state of the in-vivo monitoring camera system according to Embodiment 1 of the disclosure;

FIGS. 7A and 7B illustrate a modification example of the camera of the in-vivo imaging device according to Embodiment 1 of the disclosure, where FIG. 7A is a side view, and FIG. 7B is a top view;

FIGS. 8A and 8B illustrate a state where the camera illustrated in FIGS. 7A and 7B is gripped by a gripping portion, where FIG. 8A is a side view, and FIG. 8B is a top view;

FIGS. 9A to 9C illustrate a configuration in a case where the gripping portion has one claw portion, where FIG. 9A is a side view illustrating a state where the claw portion is open, FIG. 9B is a side view illustrating a state of the claw portion when the camera is gripped, and FIG. 9C is a sectional view of FIG. 9B;

FIGS. 10A to 10C illustrate a configuration in a case where the gripping portion has three claw portions, where FIG. 10A is a side view illustrating a state of the claw portions in a state where the gripping portion is open, FIG. 10B is a bottom view when viewed from a lower side of the gripping portion illustrated in FIG. 10A, and FIG. 10C is a side view illustrating a state of the claw portion when the camera is gripped;

FIGS. 11A to 11D illustrate a configuration in a case where the gripping portion has four claw portions, where FIG. 11A is a side view illustrating a state of the claw portions in a state where the gripping portion is open, FIG. 11B is a bottom view when viewed from the lower side of the gripping portion, FIG. 11C is a top view illustrating a configuration of the camera in the case where the gripping portion has the four claw portions, and FIG. 11D is a side view illustrating a state of the claw portions when the camera is gripped;

FIGS. 12A to 12E are schematic views illustrating a configuration of the gripping portion having an annular portion, and a method of gripping the camera which uses the gripping portion having the annular portion;

FIGS. 13A to 13C are schematic views illustrating a configuration of the gripping portion having a sucker portion, and a configuration of the camera appropriate for the gripping portion having the sucker portion;

FIGS. 14A to 14C are schematic views illustrating a configuration of the gripping portion having a pickup portion, and a method of gripping the camera which uses the gripping portion having the pickup portion;

FIGS. 15A to 15E are schematic views illustrating a support rod having a magnet disposed at the gripping portion, and a method of operating the support rod;

FIGS. 16A to 16C are schematic views illustrating a configuration of the camera whose angle is adjusted by the gripping portion;

FIGS. 17A and 17B are schematic views illustrating an example of a mechanism for maintaining a state where the gripping portion is in contact with an in-vivo side end portion;

FIGS. 18A to 18C illustrate a support rod of an in-vivo imaging device according to Embodiment 2 of the disclosure, where FIG. 18A is a schematic view illustrating respective configurations of a tubular instrument and a support rod, FIG. 18B is a side view illustrating the configuration of the support rod when viewed from the side in FIG. 18A, and FIG. 18C is a schematic view illustrating a positional relationship between the support rod and the tubular instrument when the in-vivo imaging device is used;

FIG. 19A includes a side view and a top view illustrating a configuration of a camera of the in-vivo imaging device according to Embodiment 2 of the disclosure, FIG. 19B is a schematic view illustrating a method of holding the camera which uses the support rod illustrated in FIGS. 18A to 18C, and FIG. 19C is a sectional view illustrating a configuration when the camera is gripped by a gripping portion;

FIG. 20A is a schematic view illustrating an arrangement example of support rod side electrodes in a case where the gripping portion has one claw portion, FIG. 20B is a schematic view illustrating an arrangement example of the support rod side electrodes in a case where the gripping portion has four claw portions, FIG. 20C is a schematic view illustrating an arrangement of camera side electrodes, which corresponds to an arrangement of the support rod side electrodes illustrated in FIG. 20B, and FIG. 20D is a schematic view illustrating an arrangement of the camera side electrodes, which corresponds the case where the gripping portion has the four claw portions;

FIGS. 21A to 21C are schematic views illustrating the support rod side electrode and the camera side electrode corresponding thereto in a case where the gripping portion has a sucker portion which suctions the camera;

FIGS. 22A to 22C are schematic views illustrating a waterproof mechanism of a connection portion between the support rod side electrode and the camera side electrode;

FIGS. 23A to 23D are schematic views illustrating a configuration and an operation method of a support rod and a gripping portion according to a modification example;

FIGS. 24A to 24C are schematic views illustrating a configuration and an operation method of a support rod and a gripping portion which are included in an in-vivo imaging device according to Embodiment 3 of the disclosure; and

FIG. 25 is a schematic view illustrating the support rod having a magnet disposed in the gripping portion illustrated in FIGS. 24A to 24C, and a method of operating the support rod.

### DESCRIPTION OF THE EMBODIMENTS

### Embodiment 1

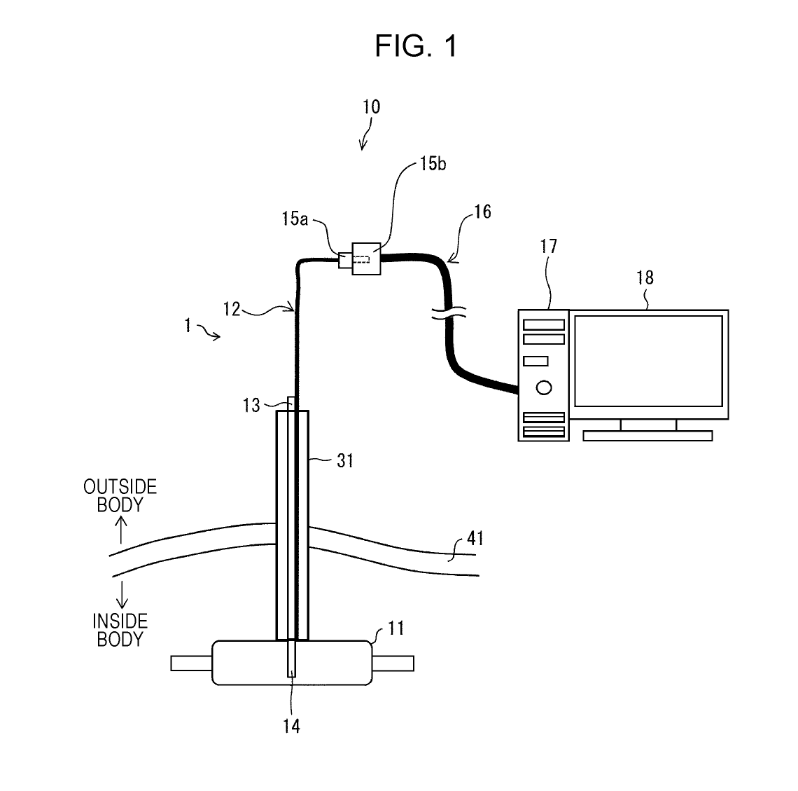

Hereinafter, an embodiment according to the disclosure will be described as follows with reference to FIGS. 1 to 17B. A shape and dimensions such as a length, a size, and a width of a configuration illustrated in each drawing do not reflect an actual shape and actual dimensions, and are appropriately modified in order to clarify and simplify the drawings.

### Configuration of In-Vivo Monitoring Camera System

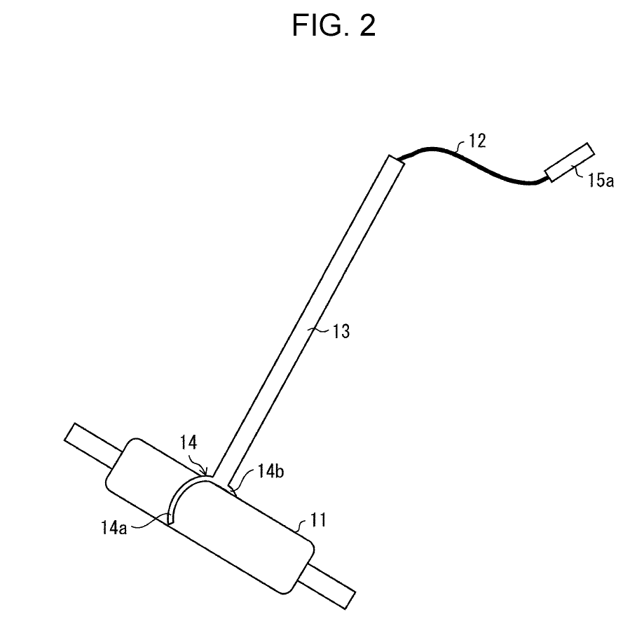

FIG. 1 is a schematic view illustrating a configuration of an in-vivo monitoring camera system 10 according to the present embodiment. FIG. 2 is a perspective view illustrating an in-vivo imaging device 1 according to the present embodiment. As illustrated in FIG. 1, the in-vivo monitoring camera system 10 includes the in-vivo imaging device 1, a control system, and an instrument side cable 16. As illustrated in FIGS. 1 and 2, the in-vivo imaging device 1 includes a camera 11 (imaging unit), a camera side cable 12, and a support rod 13 (support member) having a gripping portion 14 for gripping the camera 11. The instrument side cable 16 interconnects the camera side cable 12 and the control system to each other. The control system includes a camera control device 17 and a display 18 (display device). One end of the instrument side cable 16 is connected to the camera control device 17.

The camera side cable 12 has a projection-shaped camera side cable connector 15a on a side opposite to an end connected to the camera 11. The instrument side cable 16 has a recess-shaped instrument side cable connector 15b on a side opposite to an end connected to the camera control device 17. An operator fits the camera side cable connector 15a and the instrument side cable connector 15b to each other so as to connect the camera side cable 12 and the instrument side cable 16 to each other. A configuration may be adopted so that a recess-shaped camera side cable connector 15a and a projection-shaped instrument side cable connector are fitted to each other. FIG. 1 illustrates one pin of the camera side cable connector 15a. Here, usually, the number of pins is determined depending on the number of wires used for the cable.

The camera side cable 12 and the instrument side cable 16 are connected to each other so that the camera 11 and the camera control device 17 are electrically connected to each other. In this manner, an image captured by the camera 11 is transmitted to the camera control device 17. The camera control device 17 causes the display 18 to display the image transmitted from the camera 11, or transmits a control signal to the camera 11. The camera control device 17 and the display 18 may be integrated with each other, or may be separated from each other.

The camera side cable connector 15a is pulled outward of the body from the inside of the body through the tubular instrument 31. Therefore, an outer diameter of the camera side cable connector 15a is smaller than at least an inner diameter of the tubular instrument 31. In other words, if the outer diameter of the camera side cable connector 15a is reduced, the inner diameter of the tubular instrument 31 can be reduced, and further, a diameter of the support rod 13 can be reduced. In this manner, the in-vivo monitoring camera system 10 has an effect in that medical treatment becomes much less invasive.

The camera side cable 12 and the camera side cable connector 15a return once into the body when the camera 11 is collected. Therefore, in the instrument side cable connector 15b and the instrument side cable 16, a predetermined length portion which comes into contact with the camera side cable 12 is desired to be maintained in a cleaned state.

As illustrated in FIG. 1, the in-vivo monitoring camera system 10 employs a wired method for signal transmission between the camera 11 and the camera control device 17. In this manner, transmission speed can become faster, and a signal can be stably transmitted and received. Compared to a wireless method, lower power communication is available. A power source can be externally supplied, and thus, the camera 11 can be small in size. Therefore, the small-sized camera 11 can decrease damage when the camera 11 is introduced into the body. In this manner, an effect is achieved in that medical treatment becomes much less invasive.

In the in-vivo monitoring camera system 10, in a state where the camera 11 is held, an outer surface of the body of the gripping portion 14 of the support rod 13 is in contact with an in-vivo side end portion of the tubular instrument 31 puncturing an abdominal wall 41. The camera 11 introduced into the body is gripped by the gripping portion 14 of the support rod 13. Details of the support rod 13 will be described later. In the present embodiment, a body wall will be described as an example of the abdominal wall 41. Herein, the body wall is not limited to the abdominal wall 41.

### Configuration of Support Rod and Gripping Portion of In-Vivo Imaging Device

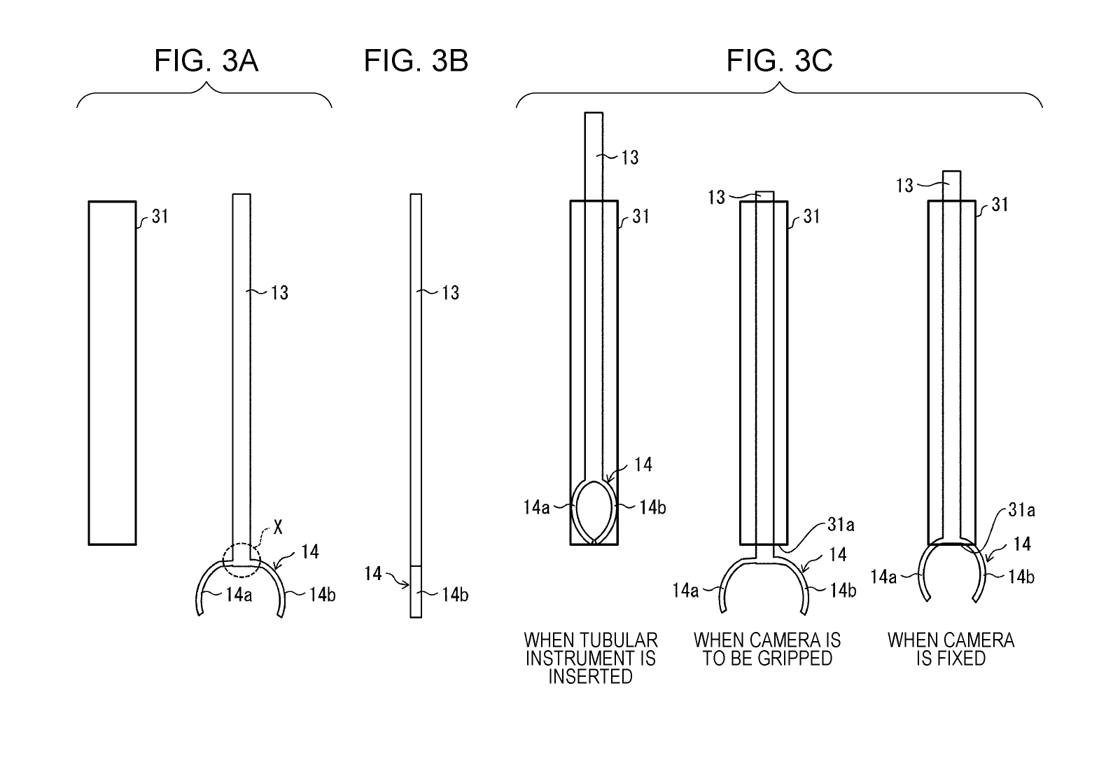

FIGS. 3A to 3C illustrate the support rod 13 and the gripping portion 14 of the in-vivo imaging device 1. FIG. 3A is a schematic view illustrating respective configurations of the tubular instrument 31, the support rod 13, and the gripping portion 14. FIG. 3B is a side view illustrating the configuration of the support rod 13 and the gripping portion 14 when viewed from the side in FIG. 3A. FIG. 3C is a schematic view illustrating a positional relationship between the support rod 13 and the tubular instrument 31 when the in-vivo imaging device 1 is used.

As illustrated in FIGS. 3A to 3C, the support rod 13 is a rod-shaped body which can be inserted into the tubular instrument 31. The in-vivo side end portion of the support rod 13 has the gripping portion 14. The gripping portion 14 has a shape for gripping the camera 11, and has two claw portions 14a and 14b. The overall dimension of the gripping portion 14 including the claw portions 14a and 14b is larger than the outer diameter of the tubular instrument 31.

In the in-vivo imaging device 1, at least a connection portion X between the support rod 13 and the gripping portion 14 is configured to include a material which is deformable if a force is applied thereto. Therefore, as illustrated in FIG. 3C, when the in-vivo imaging device 1 is used by inserting the support rod 13 into the tubular instrument 31, in a state where the gripping portion 14 is closed so that a gap decreases between the claw portions 14a and 14b, the gripping portion 14 moves into the body side (when the tubular instrument is inserted). When the gripping portion 14 moves outward of the tubular instrument 31 in order to grip the camera 11, the gripping portion 14 is brought into an open state so that the gap increases between the claw portions 14a and 14b (when the camera is gripped).

When the camera 11 is gripped by the gripping portion 14 so as to hold a position of the camera 11, the support rod 13 is pulled up outward of the body. In this manner, the claw portions 14a and 14b of the gripping portion 14 come into contact with an in-vivo side end portion 31a of the tubular instrument 31. If the support rod 13 is further pulled up, since the claw portions 14a and 14b come into contact with an in-vivo side end portion 31a of the tubular instrument 31, the force acting into the body is applied to the claw portions 14a and 14b of the gripping portion 14. As a result, since the support rod 13 is pulled up outward of the body, the gripping portion 14 is deformed so that the gap decreases between the claw portions 14a and 14b, and the gripping portion 14 grips the camera 11. In this manner, the camera 11 is gripped by the gripping portion 14 so as to hold the position of the camera 11 (when the camera is fixed).

The support rod 13 and the gripping portion 14 may be configured to include a material in which the connection portion X between the support rod 13 and the gripping portion 14 is deformable. Both the support rod 13 and the gripping portion 14 may be formed of the same material or mutually different materials. For example, in view of deformability, at least the gripping portion 14 is configured to include an elastic body such as rubber.

### Method of Installing Camera 11 in Body

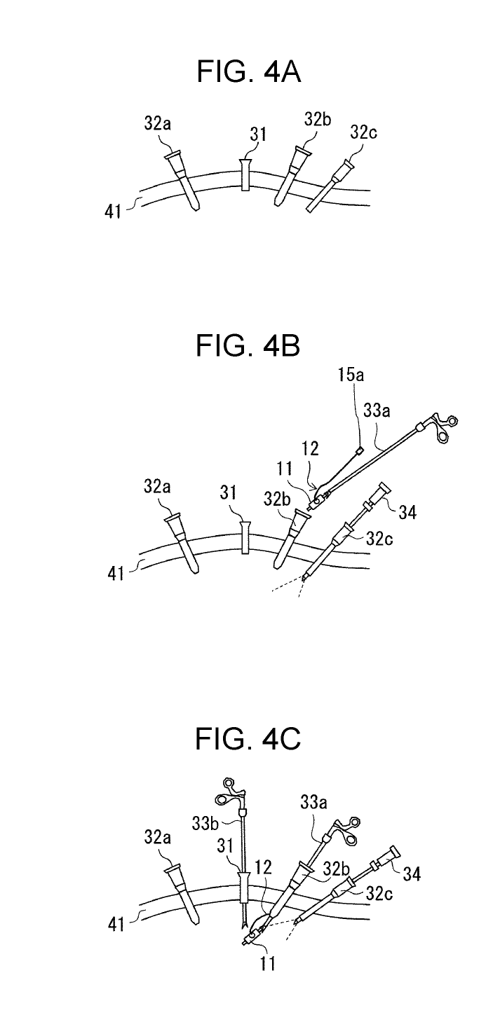

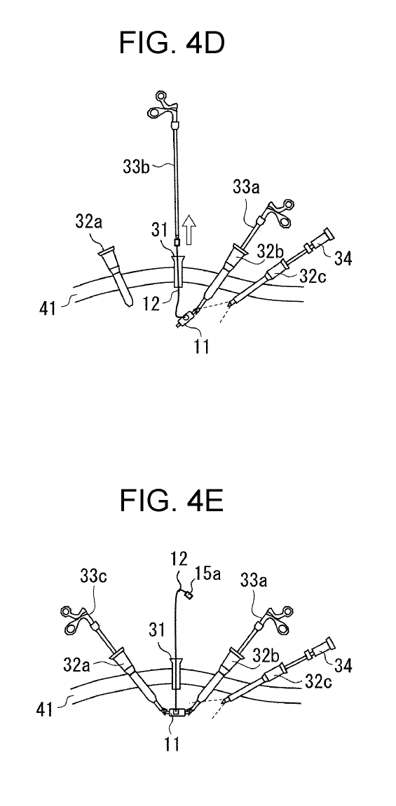

A method of installing a camera 11 of the in-vivo imaging device 1 in a body will be described. FIGS. 4A to 4E are schematic views illustrating the method of installing the camera 11 in a body according to the present embodiment.

As illustrated in FIG. 4A, an operator first opens a hole (port) for inserting forceps or an endoscope into a body cavity on the abdominal wall 41, and inserts trocars 32a to 32c into the port. Furthermore, in order to install the camera 11 inside the body cavity, the operator opens the port on the abdominal wall 41, at a position where an abdominal cavity including the lesion area can be entirely and internally viewed, and inserts the tubular instrument 31 into the port. Specifically, in a state where a needle-shaped obturator passes through the inside of the tubular instrument 31, the tubular instrument 31 is inserted into the abdominal wall 41 by causing the obturator to puncture the port position. It is preferable that the tubular instrument 31 has a smaller diameter in order to achieve less invasive medical treatment. Specifically, it is preferable that the tubular instrument 31 has the diameter of 3 mm or shorter. After at least one of the trocars 32a to 32c is inserted, the operator sends gas into the body through the trocars 32a to 32c, and widens the body cavity in advance so as to secure a space for inserting instruments.

Next, as illustrated in FIG. 4B, the operator inserts an endoscope 34 into the body cavity through the trocar 32c. While the operator observes the inside of the body by using the endoscope 34, the operator inserts the camera 11, the camera side cable 12, and the camera side cable connector 15a which are gripped using forceps 33a into the body cavity through the trocar 32b.

Next, as illustrated in FIG. 4C, the operator operates the forceps 33a so as to move the camera 11 to the vicinity of the tubular instrument 31, and inserts the forceps 33b into the body cavity through the tubular instrument 31.

Next, as illustrated in FIG. 4D, in a state where the forceps 33b pinch the camera side cable 12, the operator pulls out the forceps 33b from the tubular instrument 31 so that the camera side cable 12 is pulled out of the body. In this case, (the gripping portion of) the camera 11 is in a gripped state using the forceps 33a. An example is illustrated in which the camera 11 side is inserted into the body cavity. Herein, a procedure may be used as follows. The camera 11 side cable connector 15a is first inserted into the body cavity. While the camera side cable connector 15a is gripped using the forceps, the camera 11 may be inserted into the body.

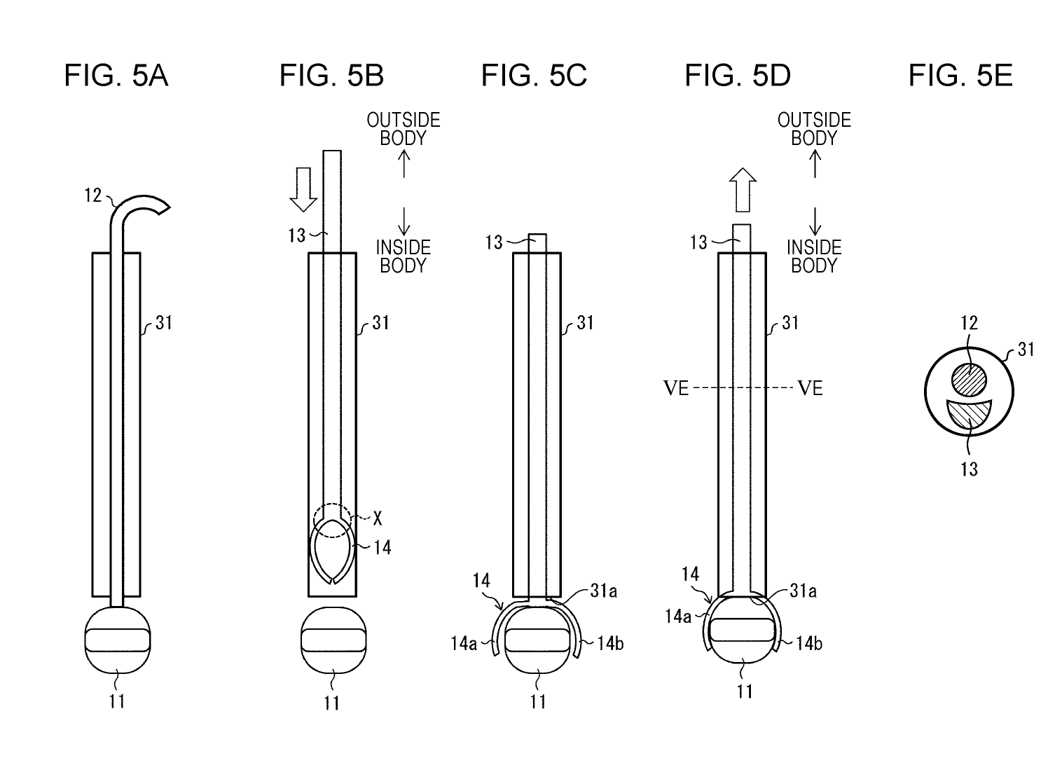

Next, as illustrated in FIG. 4E, the operator pulls up the camera side cable 12 pulled outward of the body by using the forceps or the operator's hand. The operator inserts the forceps 33c into the body through the trocar 32a, and grips both ends of the camera 11 inside the body by using the forceps 33a and 33c so as to preliminarily hold the camera 11. In the present embodiment, after the camera side cable 12 is pulled up, the support rod 13 is used so as to hold the position of the camera 11. FIGS. 5A to 5E are schematic views illustrating a method of holding the camera 11 by using the support rod 13 according to the present embodiment, and illustrate the method of installing a camera in a body after a step in FIG. 4E. FIG. 5A illustrates a step in FIG. 4E. FIGS. 5B to 5D illustrate a step after the step in FIG. 4E. FIG. 5E is a sectional view taken along line VE-VE in FIG. 5D. In order to simplify the drawing, the illustration of the camera side cable 12 is omitted in FIGS. 5B to 5D.

After the camera side cable 12 is pulled up using the forceps or the hand (corresponding to FIG. 5A), as illustrated in FIG. 5B, the operator inserts the support rod 13 into the body cavity through the tubular instrument 31 by using the forceps or the hand. Here, as described above, at least the connection portion X between the support rod 13 and the gripping portion 14 is configured to include the deformable material. Accordingly, the gripping portion 14 is inserted into the tubular instrument 31 in a closed state so that the gap decreases between the claw portions 14a and 14b.

The gripping portion 14 is further moved to the inside of the body, and is located in the vicinity of the camera 11. In this case, as illustrated in FIG. 5C, the gripping portion 14 is in a state where the claw portions 14a and 14b are largely open, and can grip the camera 11. The operator pushes down the support rod 13 so that the claw portions 14a and 14b of the gripping portion 14 come into contact with the camera 11.

Next, as illustrated in FIG. 5D, the operator pulls up the support rod 13 outward of the body in a state where the gripping portion 14 is open. In this case, the gripping portion 14 comes into contact with the in-vivo side end portion 31a of the tubular instrument 31. If the support rod 13 is further pulled up, the gripping portion 14 is deformed so that the gap decreases between the claw portions 14a and 14b. Finally, the camera 11 is gripped by the claw portions 14a and 14b of the gripping portion 14, and is held at a position in the vicinity of the in-vivo side end portion 31a of the tubular instrument 31.

In the in-vivo imaging device according to the present embodiment, the tubular instrument 31 is not particularly limited as long as the tubular instrument 31 has a tubular structure which can be inserted into the body. For example, as the tubular instrument 31, the trocar may be used. Many trocars can be used as the tubular instrument 31. However, in some cases, it may not be appropriate to use the in-vivo side end portion of the trocar in coming into contact with the in-vivo side end portion 31a of the tubular instrument 31 in order to grip the camera 11 by deforming the gripping portion 14. In this case, instead of the tubular instrument 31, it is also possible to use an outer tube whose in-vivo side end portion has a horizontal shape. It is also possible to grip the camera 11 by using the tubular instrument 31 together with the outer tube and deforming the gripping portion 14.

Here, as illustrated in FIG. 5E, in view of a space occupied by the camera side cable 12 inside the tubular instrument 31, the design of the support rod 13 may be changed to have a semi-circular cross-sectional shape. That is, the support rod 13 has a semi-circular rod shape.

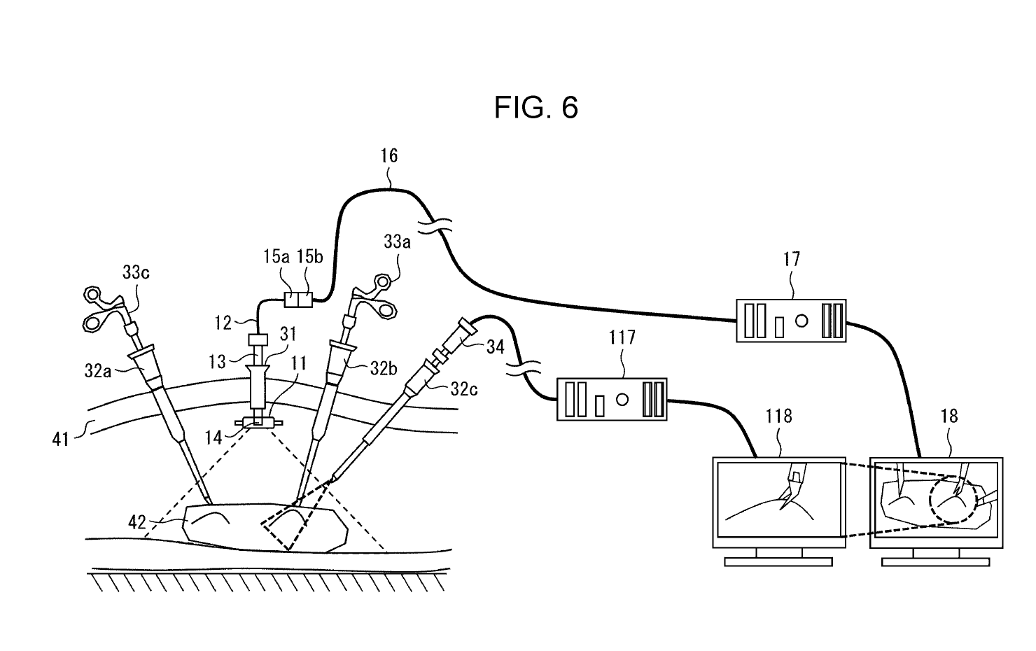

After the camera 11 is installed inside the body, as illustrated in FIG. 6, the camera side cable connector 15a is fitted into the instrument side cable connector 15b so as to connect the camera side cable 12 and the instrument side cable 16 to each other. In this manner, a localized image of a treatment site is displayed on a display 118 by an endoscope control device 117. An entire image inside the abdominal cavity including an organ 42 captured by the camera 11 is displayed on the display 18 by the camera control device 17.

A procedure after the use is as follows. In a state where the operator causes the forceps 33a to grip the gripping portion of the camera 11 located inside the body, the operator pushes down the support rod 13, and separates the support rod 13 and the camera 11 from each other. The operator pulls the support rod 13 away from the tubular instrument 31. Thereafter, the operator pulls the camera 11 and the camera side cable 12 from the trocar 32b outward of the body, and pulls the support rod 13 from the tubular instrument 31 outward of the body.

### Modification Example of Camera

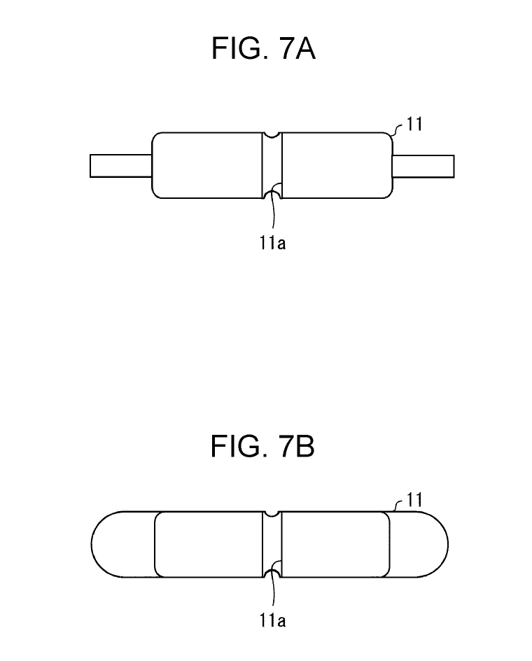

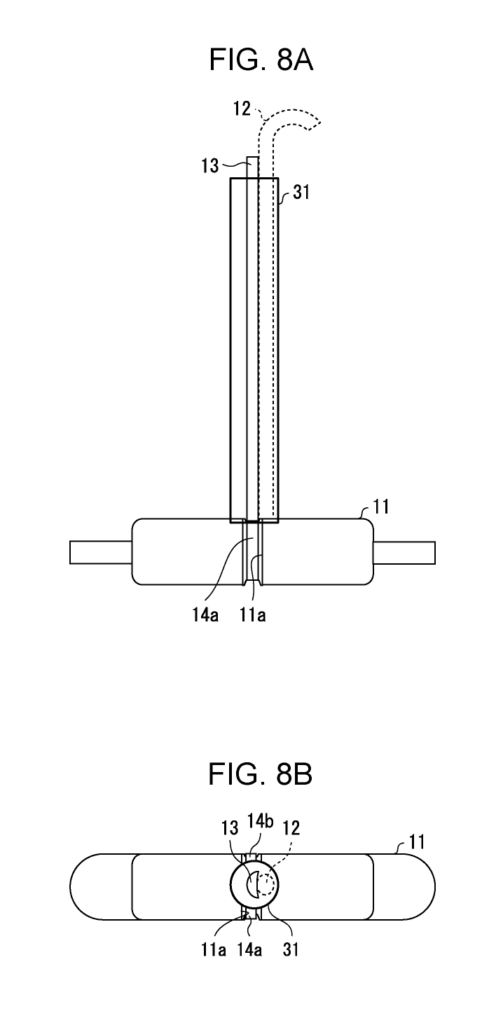

FIGS. 7A and 7B illustrate a modification example of the camera 11. FIG. 7A is a side view, and FIG. 7B is a top view. FIGS. 8A and 8B illustrate a state where the camera 11 illustrated in FIGS. 7A and 7B is gripped by the gripping portion 14. FIG. 8A is a side view, and FIG. 8B is a top view.

As illustrated in FIGS. 7A and 7B, the in-vivo imaging device 1 may have a structure in which a groove 11a is formed in the camera 11. As illustrated in FIGS. 8A and 8B, the groove 11a is disposed at a position corresponding to the claw portions 14a and 14b when the camera 11 is gripped by the gripping portion 14. Therefore, when the camera 11 is gripped by the gripping portion 14, the claw portions 14a and 14b are respectively located inside the groove 11a of the camera 11. Thus, separation of the camera 11 from the gripping portion 14 can be avoided.

### Modification Example of Gripping Portion

In the example of FIGS. 3A to 3C, the gripping portion 14 has two claw portions 14a and 14b. However, the number of claw portions of the gripping portion 14 in the present embodiment is not limited to two. If the camera 11 can be gripped, the number of the claw portions is not limited.

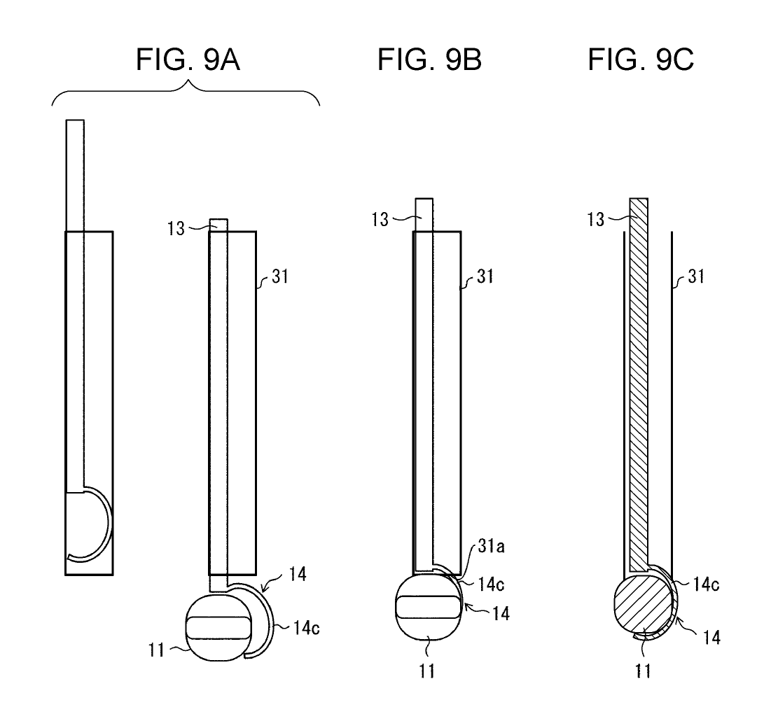

FIGS. 9A to 9C illustrate a configuration in a case where the gripping portion 14 has one claw portion 14c. FIG. 9A is a side view illustrating a state where the claw portion 14c is open, FIG. 9B is a side view illustrating a state of the claw portion 14c when the camera 11 is gripped, and FIG. 9C is a sectional view of FIG. 9B. As illustrated in FIGS. 9A to 9C, in the case where the gripping portion 14 has one claw portion 14c, the claw portion 14c has a shape which is bent so as to be caught on an outer end portion of the body in the camera 11. A dimension of the claw portion 14c is larger than the outer diameter of the tubular instrument 31. Therefore, if the support rod 13 is pulled up in a state where the claw portion 14c is open inside the body cavity, as illustrated in FIG. 9B, the claw portion 14c comes into contact with the in-vivo side end portion 31a of the tubular instrument 31. If the support rod 13 is further pulled up, the claw portion 14c is deformed so as to surround the camera 11, and finally grips the camera 11.

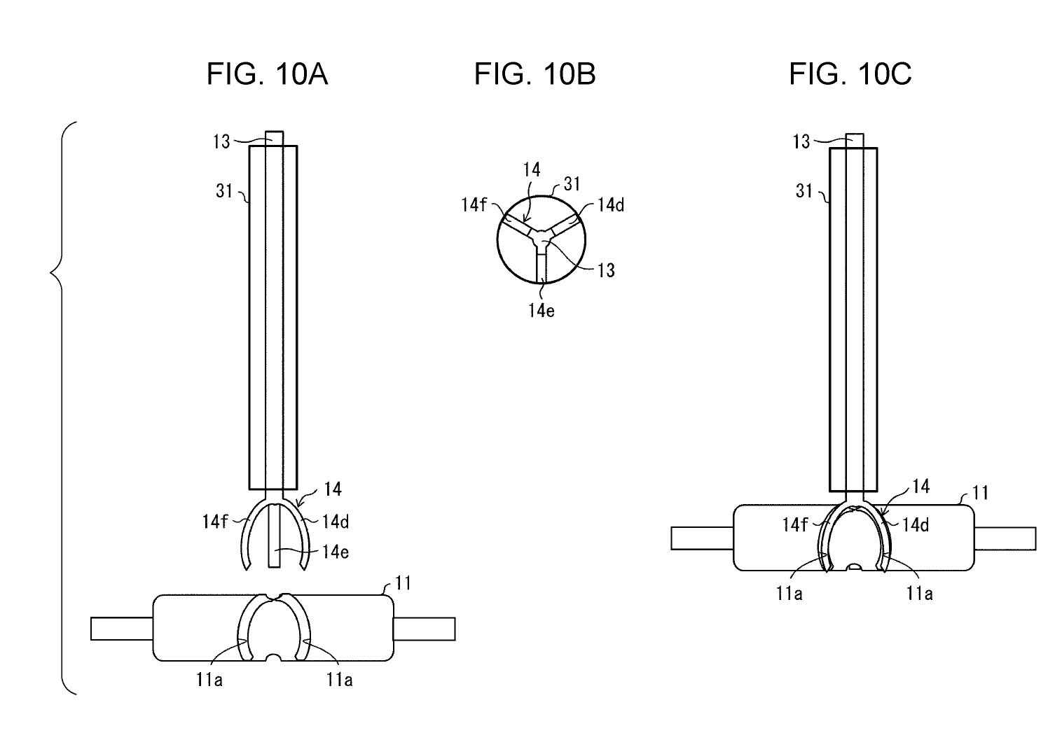

FIGS. 10A to 10C illustrate a configuration in a case where the gripping portion 14 has three claw portions 14d to 14f. FIG. 10A is a side view illustrating a state where the claw portions 14d to 14f are open. FIG. 10B is a bottom view when the gripping portion 14 illustrated in FIG. 10A is viewed from the lower side. FIG. 10C is a side view illustrating a state of the claw portions 14d to 14f when the camera 11 is gripped. As illustrated in FIGS. 10A to 10C, the gripping portion 14 may have the three claw portions 14d to 14f. In this case, separation of the camera 11 from the gripping portion 14 can be avoided, if three grooves 11a are formed in the camera 11 so as to correspond to the claw portions 14d to 14f.

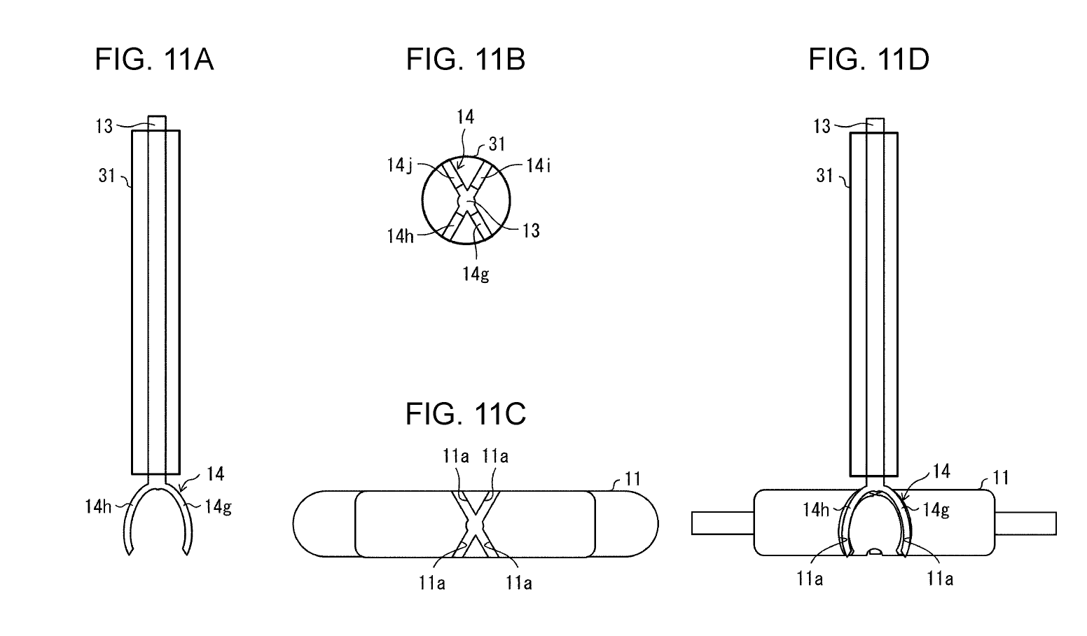

FIGS. 11A to 11D illustrate a configuration in a case where the gripping portion 14 has four claw portions 14g to 14j. FIG. 11A is a side view illustrating a state where the claw portions 14g to 14j are open. FIG. 11B is a bottom view when the gripping portion 14 illustrated in FIG. 11A is viewed from the lower side. FIG. 11C is a top view illustrating a configuration of the camera 11 in the case where the gripping portion 14 has the four claw portions 14g to 14j. FIG. 11D is a side view illustrating a state of the claw portions 14g to 14j when the camera 11 is gripped. As illustrated in FIGS. 11A, 11B, and 11D, the gripping portion 14 may have the four claw portions 14g to 14j. In this case, as illustrated in FIG. 11C, the camera 11 has four grooves 11a formed so as to correspond to the claw portions 14g to 14j. In this manner, separation of the camera 11 from the gripping portion 14 can be avoided.

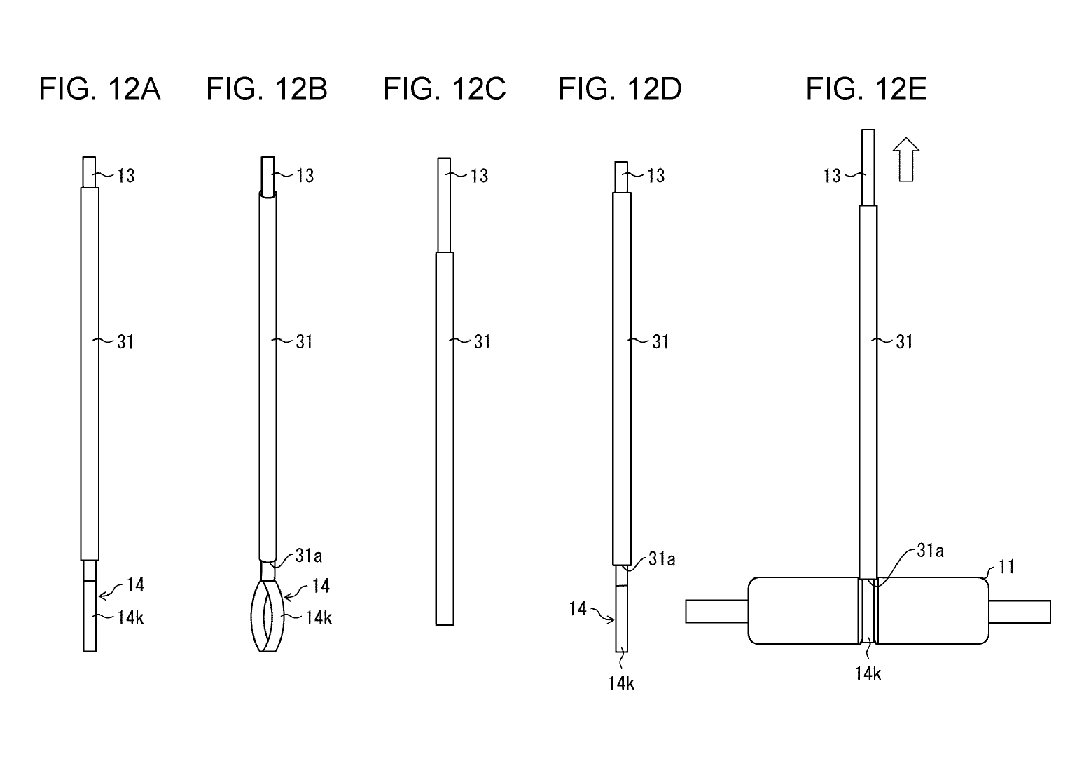

The gripping portion 14 is not limited to a configuration having the claw portion. For example, as illustrated in FIGS. 12A and 12B, the gripping portion 14 may have an annular portion 14k. In this case, the inner diameter of the annular portion 14k is slightly larger than the dimension of the camera 11. That is, a configuration is adopted so that the camera 11 can enter the inside of the annular portion 14k in an open state. According to an actual method of installing the camera 11, as illustrated in FIG. 12C, the operator first inserts the support rod 13 into the tubular instrument 31, and moves the gripping portion 14 into the body side. As illustrated in FIG. 12D, the operator further moves the gripping portion 14 into the body side so as to bring the annular portion 14k into an open state. In this case, the operator operates the camera 11 so as to enter the inside of the annular portion 14k in the open state. As illustrated in FIG. 12E, when the support rod 13 is pulled up, the annular portion 14k comes into contact with the in-vivo side end portion 31a of the tubular instrument 31. In this manner, the annular portion 14k is deformed so as to decrease the inner diameter, and is brought into a closed state. The annular portion 14k is closed by pulling up the support rod 13 in this way. Accordingly, the camera 11 is gripped inside the annular portion 14k.

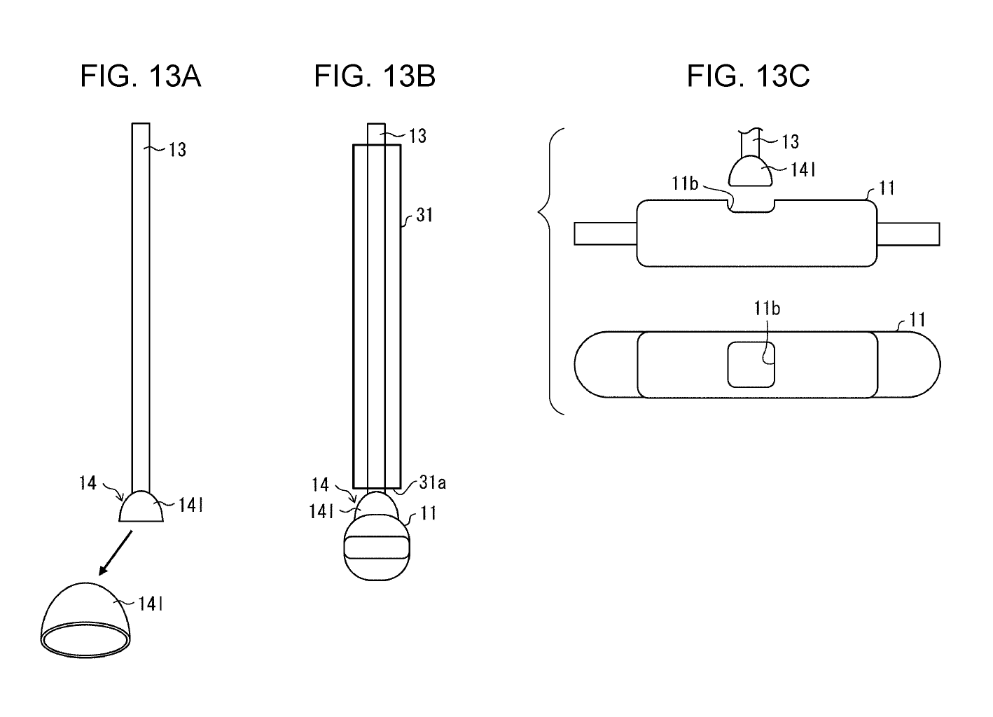

As illustrated in FIGS. 13A to 13C, the gripping portion 14 may have a sucker portion 14l which suctions the camera 11. In this case, as illustrated in FIG. 13B, the sucker portion 14l may have a shape appropriate for a shape of the camera 11. As illustrated in FIG. 13C, the camera 11 may have a recess portion 11b appropriate for the sucker portion 14l. In a case where the gripping portion 14 has the sucker portion 14l, the sucker portion 14l has a function of holding the camera 11. Therefore, when the support rod 13 is pulled up outward of the body, the gripping portion 14 is not deformed by coming into contact with the in-vivo side end portion 31a of the tubular instrument 31.

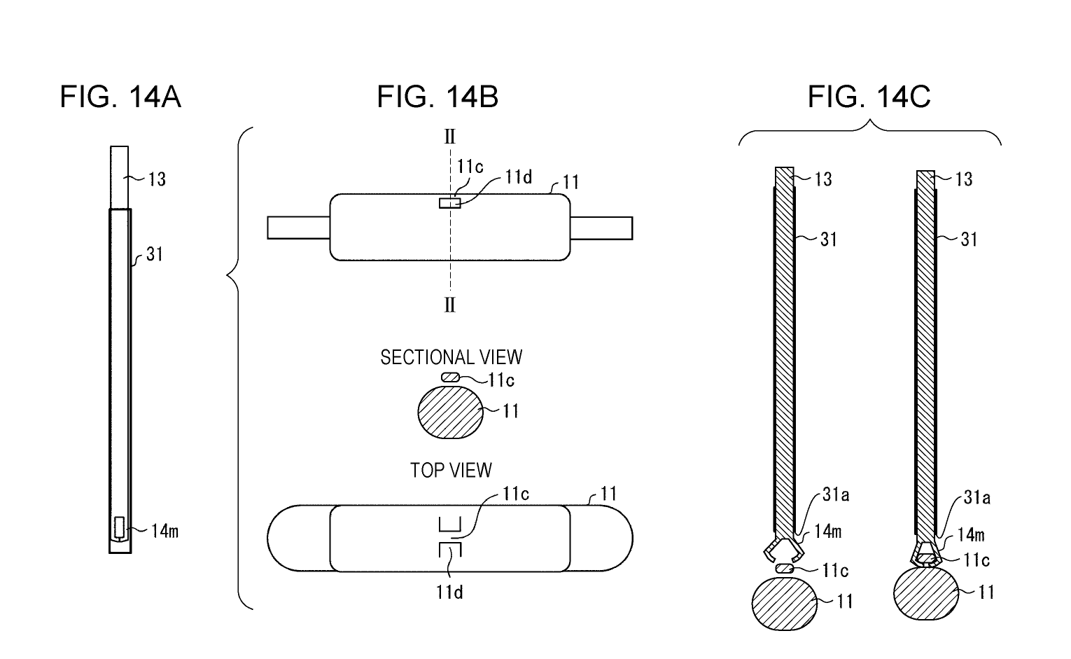

As illustrated in FIGS. 14A to 14C, the camera 11 may have a pickup portion 11c. As illustrated in FIG. 14B, an upper surface of the pickup portion 11c is flush with an upper surface of the camera 11. A cavity portion 11d is disposed on the lower side of the pickup portion 11c. As illustrated in the top view in FIG. 14B, a bottom surface of the cavity portion 11d is an inclined surface inclined inward in a short direction of the camera 11.

As illustrated in FIGS. 14A to 14C, the gripping portion 14 is configured to pick up the pickup portion 11c. More specifically, the gripping portion 14 has a forceps portion 14m similar to a distal end shape of the forceps. In the short direction of the camera 11, the forceps portion 14m in an open state is smaller than the dimension of the camera 11, and is larger than the pickup portion 11c and the tubular instrument 31. Therefore, if the support rod 13 is pulled up in a state where the forceps portion 14m is open inside the body cavity, as illustrated in the right drawing in FIG. 14C, the forceps portion 14m comes into contact with the in-vivo side end portion 31a of the tubular instrument 31. If the support rod 13 is further pulled up, the forceps portion 14m enters the cavity portion 11d of the camera 11. In this manner, the forceps portion 14m is deformed so as to pick up the pickup portion 11c, and the camera 11 is finally gripped. According to the configuration illustrated in FIGS. 14A to 14C, the gripping portion 14 has such a dimension that allows the gripping portion 14 to pick up the pickup portion 14m which is smaller than the dimension of the camera 11. Therefore, miniaturization and diameter reduction of the gripping portion 14 can be realized.

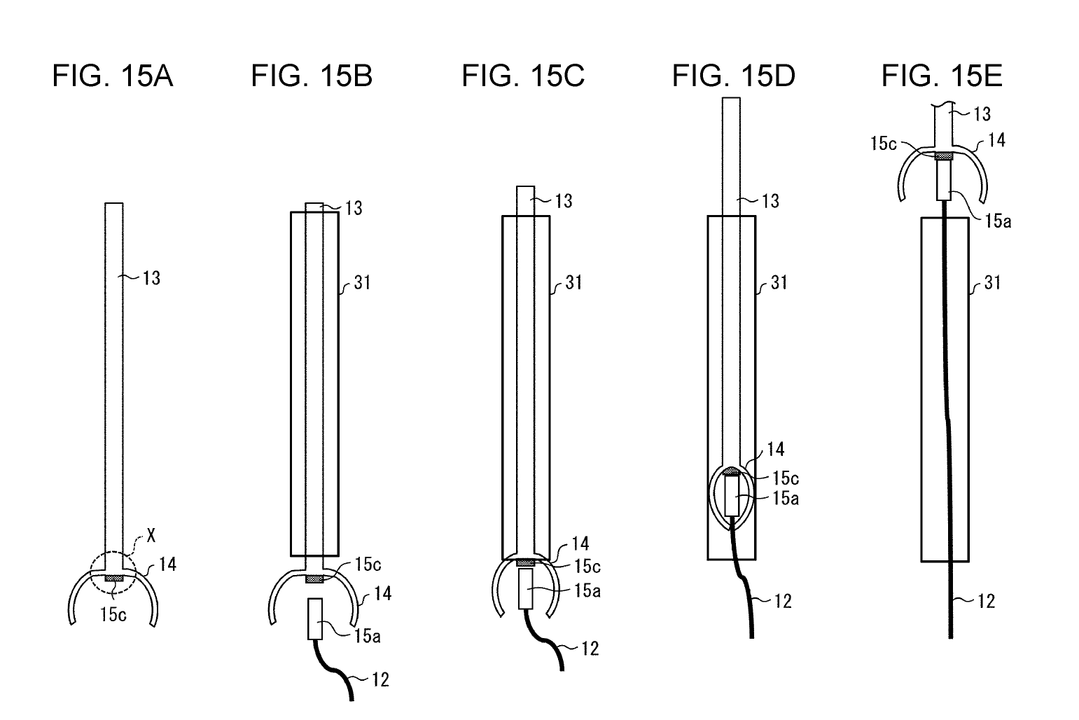

As illustrated in FIG. 15A, the gripping portion 14 may have a magnet 15c or a magnetic substance in the connection portion X connected to the support rod 13. In this case, the magnet or the magnetic substance is attached to the distal end portion of the camera side cable connector 15a, and the magnet 15c magnetically attracts the camera side cable connector 15a. Since the magnet 15c is provided in this way, the support rod 13 having the gripping portion 14 can be used as a drawing tool for pulling up the camera side cable 12. This drawing tool is used for a step in FIG. 4D, for example.

FIGS. 15B to 15E are schematic views illustrating a method of pulling up the camera side cable 12 by using the support rod 13 illustrated in FIG. 15A. As illustrated in FIG. 15B, the operator first inserts the support rod 13 into the tubular instrument 31, and locates the support rod 13 in the vicinity of the camera side cable connector 15a inside the body cavity in a state where the gripping portion 14 is open. As illustrated in FIG. 15C, the magnet 15c disposed in the gripping portion 14 is caused to magnetically attract the camera side cable connector 15a. As illustrated in FIG. 15D, in a state where the magnet 15c magnetically attracts the camera side cable connector 15a, the support rod 13 is pulled out of the tubular instrument 31 so as to pull the camera side cable 12 outward of the body. In this case, since the support rod 13 is pulled up, the gripping portion 14 comes into contact with the tubular instrument 31, and is brought into a closed state. Therefore, the camera side cable 12 or the camera side cable connector 15a is pulled up inside the tubular instrument 31 in a state of being gripped by the gripping portion 14.

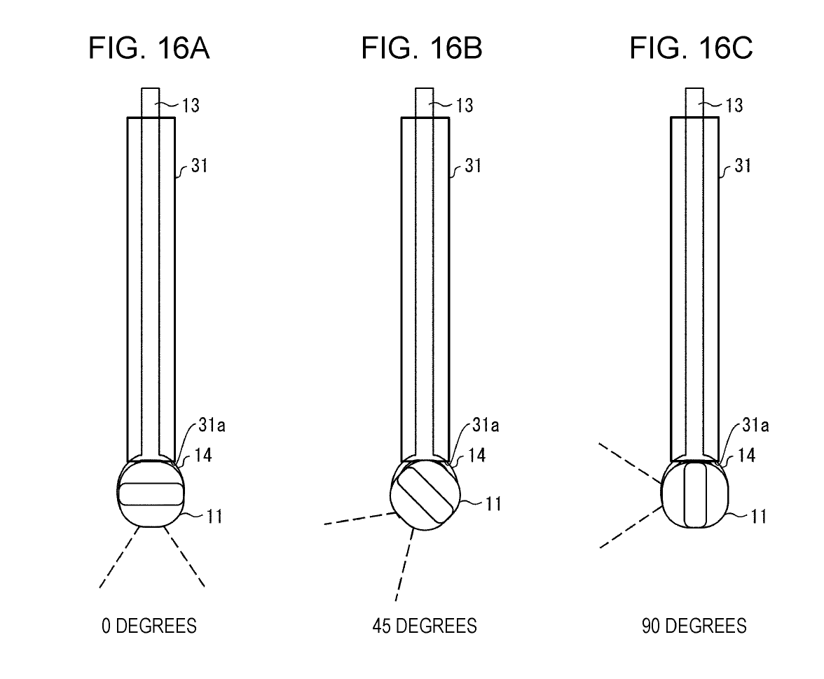

According to the present embodiment, as illustrated in FIGS. 16A to 16C, it is possible to adjust a holding angle of the camera 11. For example, in a step in FIG. 4E, the camera 11 is preliminarily held at a desired angle by using the forceps 33a and 33c. Since the preliminarily held camera 11 is held by using the support rod 13 (steps in FIGS. 5B to 5D), it is possible to adjust the holding angle of the camera 11.

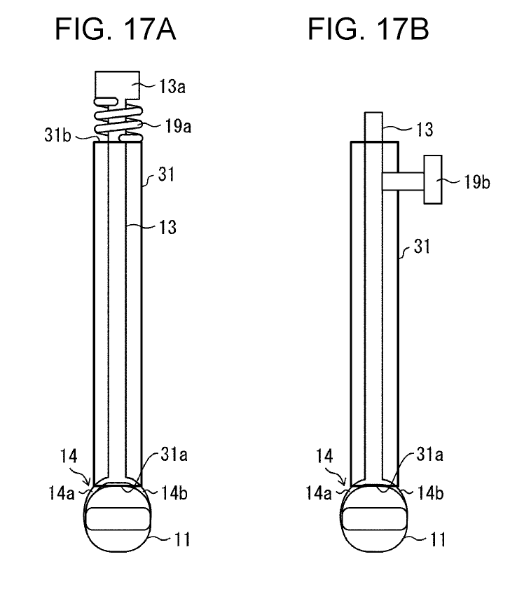

Here, the gripping portion 14 gripping the camera 11 maintains a state of being in contact with the in-vivo side end portion 31a of the tubular instrument 31. In this manner, the position of the camera 11 can be more stably held. FIGS. 17A and 17B are schematic views illustrating an example of a mechanism for maintaining a state where the gripping portion 14 is in contact with the in-vivo side end portion 31a.

According to a configuration illustrated in FIG. 17A, the in-vivo imaging device includes a spring 19a which biases the support rod 13 inserted into the tubular instrument 31 against the outside of the body. The support rod 13 is located so as to be insertable into the spring 19a. A contact portion 13a which comes into contact with an end portion of the spring 19a and locks the spring 19a is disposed in an outer end portion of the body of the support rod 13. An end portion (in-vivo side end portion) opposite to the contact portion 13a in the spring 19a is in contact with an outer end portion 31b in the tubular instrument 31. In this manner, the spring 19a presses the contact portion 13a which is the outer end portion of the body in the support rod 13. Therefore, the claw portions 14a and 14b of the gripping portion 14 maintain a state of being in contact with the in-vivo side end portion 31a of the tubular instrument 31.

According to a configuration illustrated in FIG. 17B, a stopper 19b is disposed in the tubular instrument 31. The stopper 19b has a structure for locking the support rod 13 inserted into the tubular instrument 31 in moving outward of the body or moving inward of the body. Therefore, since the stopper 19b locks the support rod 13, the claw portions 14a and 14b of the gripping portion 14 maintain a state of being in contact with the in-vivo side end portion 31a of the tubular instrument 31.

### Embodiment 2

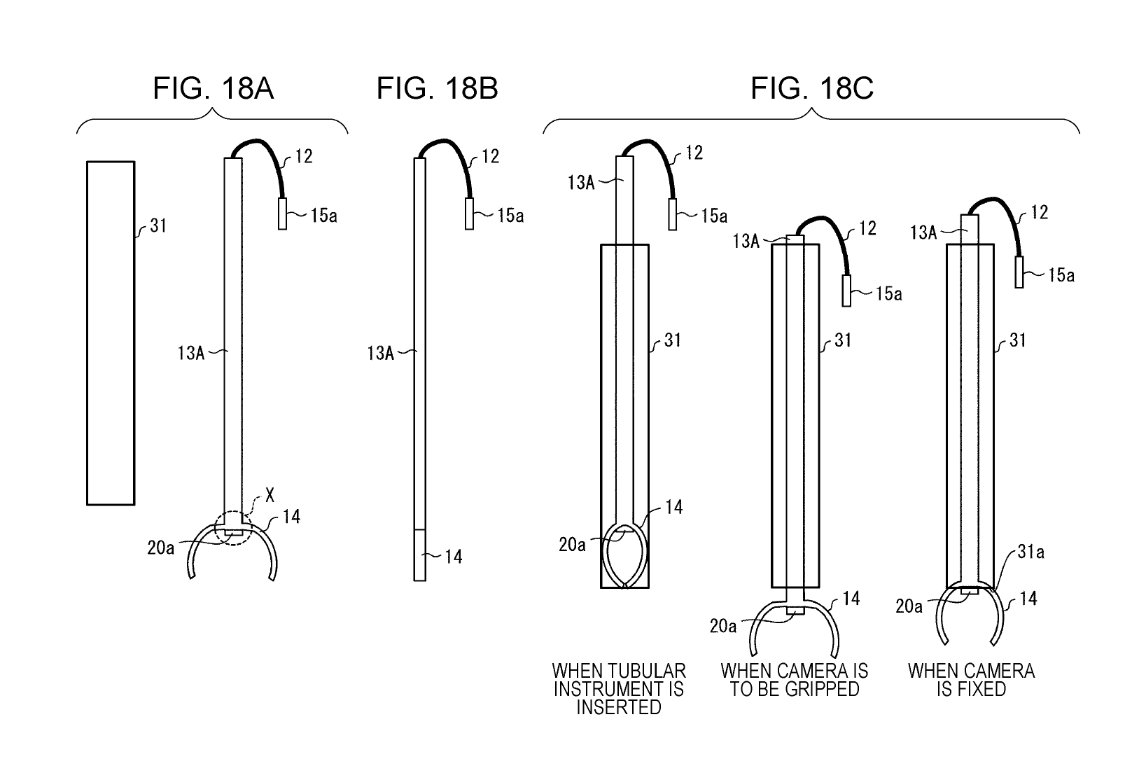

Referring to FIGS. 18A to 23D, another embodiment according to the disclosure will be described as follows. For the convenience of description, the same reference numerals will be given to members having functions which are the same as those of the members described in the above embodiment, and description thereof will be omitted.

FIGS. 18A to 18C illustrate a support rod 13A and the gripping portion 14 of the in-vivo imaging device according to the present embodiment. FIG. 18A is a schematic view illustrating respective configurations of the tubular instrument 31, the support rod 13A, and the gripping portion 14. FIG. 18B is a side view illustrating the configuration of the support rod 13A and the gripping portion 14 when viewed from the side in FIG. 18A. FIG. 18C is a schematic view illustrating a positional relationship between the support rod 13A and the tubular instrument 31 when the in-vivo imaging device is used.

As illustrated in FIGS. 18A and 18B, the in-vivo imaging device according to the present embodiment is different from that according to Embodiment 1 in that the support rod 13A internally includes the camera side cable 12. A support rod side electrode 20a (support member side electrode) connected to the camera side cable 12 is disposed in the connection portion X between the support rod 13A and the gripping portion 14.

As illustrated in FIG. 18C, when the support rod 13A is inserted into the tubular instrument 31, the gripping portion 14 moves into the body side in a state where the gripping portion 14 is closed so as to decrease the gap between the claw portions 14a and 14b (when the tubular instrument 31 is inserted). In this case, the support rod side electrode 20a is blocked from the body cavity by the claw portions 14a and 14b.

When the gripping portion 14 moves outward of the tubular instrument 31 in order to grip the camera 11, the gripping portion 14 is brought into an open state so as to increase the gap between the claw portions 14a and 14b (when the camera is gripped). In this case, the support rod side electrode 20a is exposed inside the body cavity, since the claw portions 14a and 14b are brought into an open state.

The claw portions 14a and 14b of the gripping portion 14 are brought into contact with the in-vivo side end portion 31a of the tubular instrument 31 by pulling up the support rod 13A outward of the body, and the camera 11 is gripped by the gripping portion 14 (when the camera is fixed). In this case, the support rod side electrode 20a comes into contact with the camera 11, and is electrically connected to the camera 11.

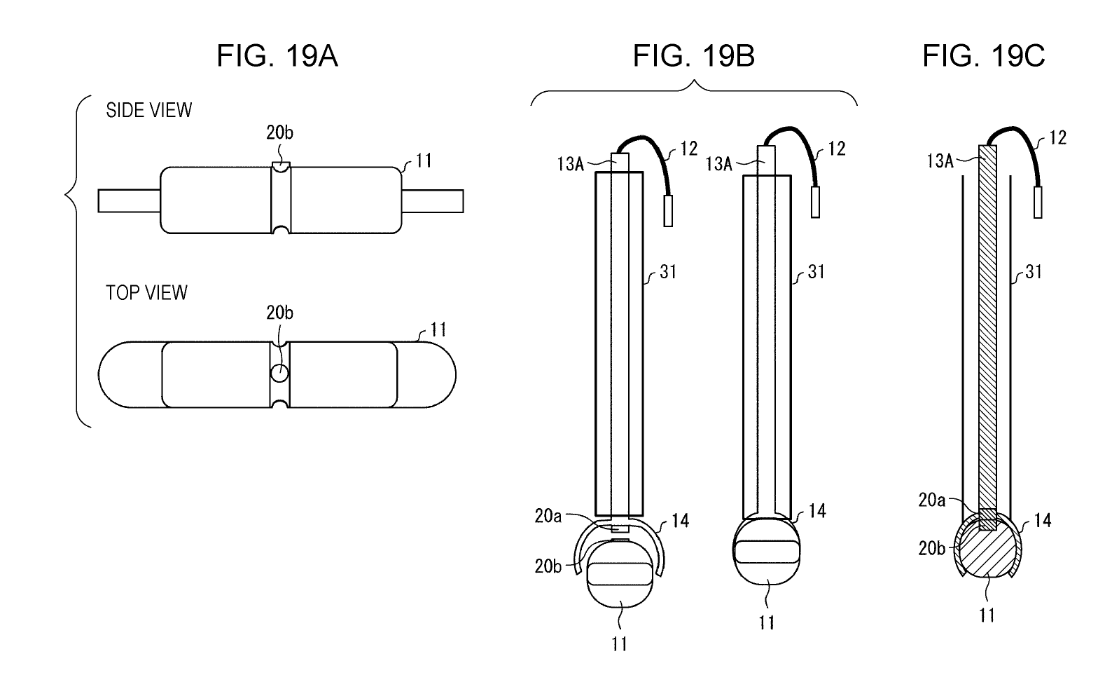

FIG. 19A is a side view and a top view illustrating a configuration of the camera 11 of the in-vivo imaging device according to the present embodiment. FIG. 19B is a schematic view illustrating a method of holding the camera 11 by using the support rod 13A. FIG. 19C is a sectional view illustrating a configuration when the camera 11 is gripped by the gripping portion 14. As illustrated in FIG. 19A, an upper surface of the camera 11 is provided with a camera side electrode 20b (imaging unit side electrode) so as to correspond to the support rod side electrode 20a disposed in the gripping portion 14.

As illustrated in FIG. 19B, when the gripping portion 14 is moved into the body side through the tubular instrument 31 and the claw portions 14a and 14b of the gripping portion 14 are brought into an open state, the operator moves the camera 11 so that the camera side electrode 20b faces the support rod side electrode 20a. The support rod 13A is pulled up outward of the body, and the camera 11 is gripped by the gripping portion 14. In this case, as illustrated in FIG. 19C, the support rod side electrode 20a and the camera side electrode 20b are connected to each other. As a result, according to the present embodiment, when the camera 11 is gripped by the gripping portion 14, the camera 11 and the camera side cable 12 are electrically connected to each other.

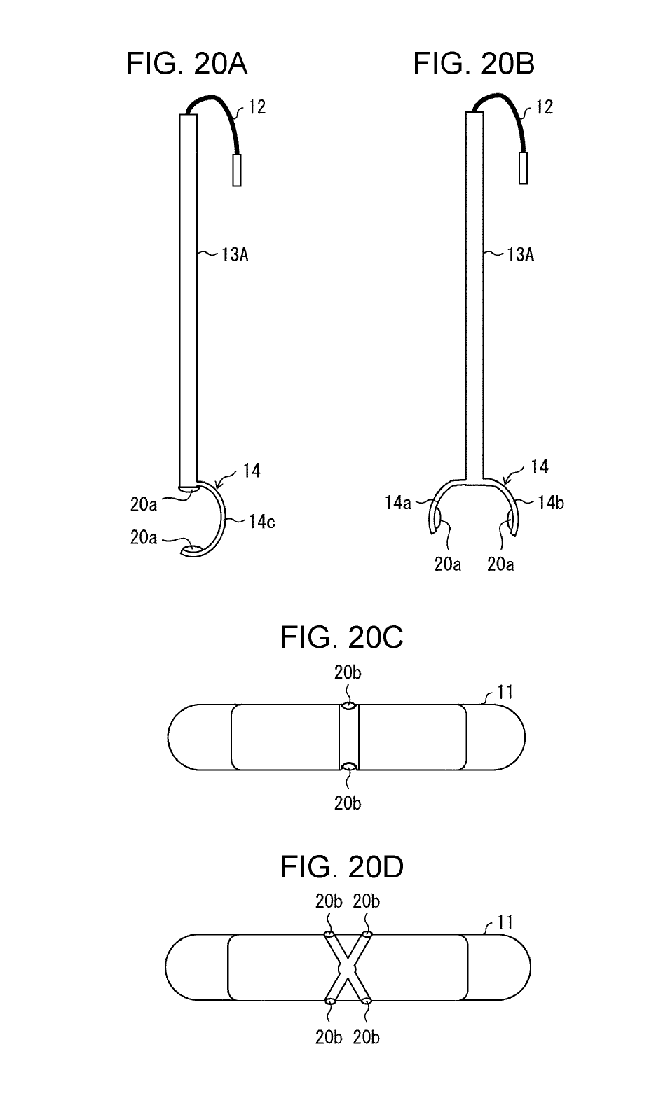

In an example illustrated in FIGS. 19A to 19C, the support rod side electrode 20a is disposed in the connection portion X between the support rod 13A and the gripping portion 14. However, a position and the number of the support rod side electrodes 20a are not limited to the example in FIGS. 19A to 19C. For example, at least two support rod side electrodes 20a may be provided as illustrated in FIGS. 20A and 20B.

FIG. 20A illustrates an example in a case where the gripping portion 14 has one claw portion 14c. In this case, the two support rod side electrodes 20a are respectively a power electrode and a ground electrode, and are located so as to be separated from each other in an end portion in an upward-downward direction (direction from the inside of the body toward the outside of the body) in the claw portion 14c. FIG. 20B illustrates an example in a case where the gripping portion 14 has two claw portions 14a and 14b. In this case, the two support rod side electrodes 20a are respectively the power electrode and the ground electrode, and are located so as to be separated from each other in the claw portions 14a and 14b. In this way, short-circuit is avoided by arranging the power electrode and the ground electrode as the two support rod side electrodes 20a so as to be separated from each other.

As illustrated in FIGS. 20C and 20D, the camera side electrode 20b disposed in the camera 11 may be located at a position where the camera side electrode 20b comes into contact with the support rod side electrode 20a when the camera 11 is gripped by the gripping portion 14. In an example illustrated in FIG. 20C, the camera side electrode 20b is located so as to correspond to the location of the support rod side electrode 20a illustrated in FIG. 20B. In an example illustrated in FIG. 20D, the camera side electrode 20b is located so as to correspond to a case where the gripping portion has four the claw portions. In a case of the configuration illustrated in FIG. 20D, four support rod side electrodes 20a are provided, and are located so as to be separated from each other in the four claw portions.

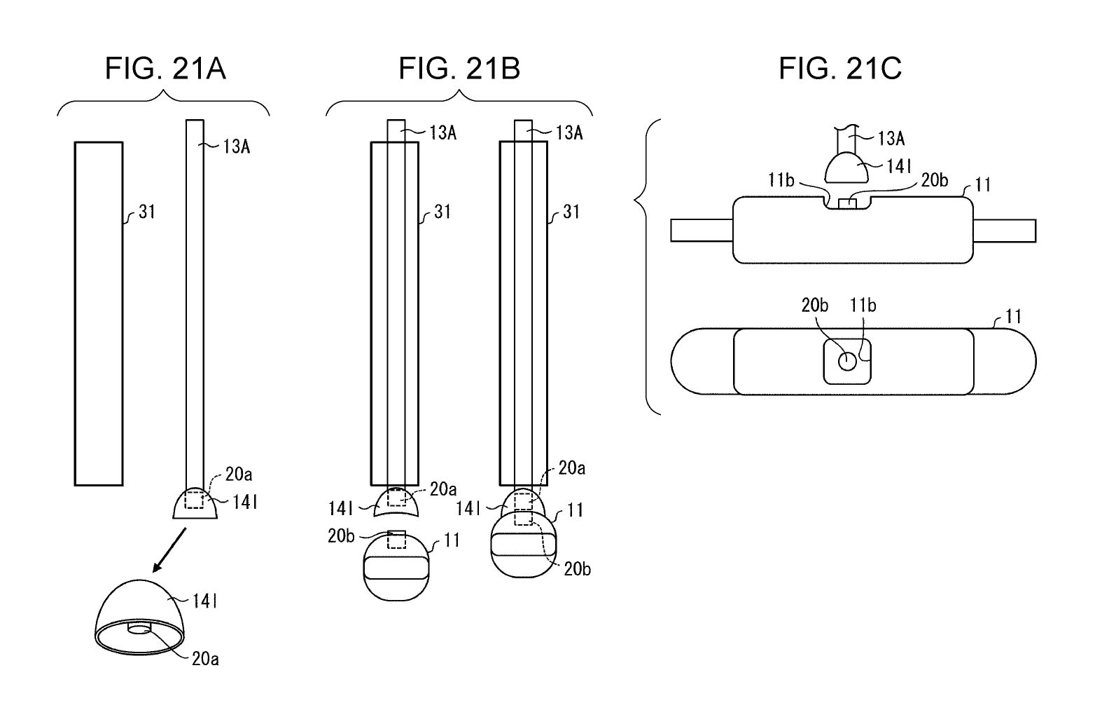

As illustrated in FIGS. 21A to 21C, in a case where the gripping portion 14 has a sucker portion 14l which suctions the camera 11, the support rod side electrode 20a is located inside the sucker portion 14l. As illustrated in FIG. 21B, in a case where the sucker portion 14l has a shape appropriate for the shape of the camera 11, the camera side electrode 20b is disposed at a position where the camera side electrode 20b comes into contact with the support rod side electrode 20a when the camera 11 is gripped by the gripping portion 14. As illustrated in FIG. 21C, in a case where the camera 11 has a recess portion 11b appropriate for the sucker portion 14l, the camera side electrode 20b is disposed inside the recess portion 11b.

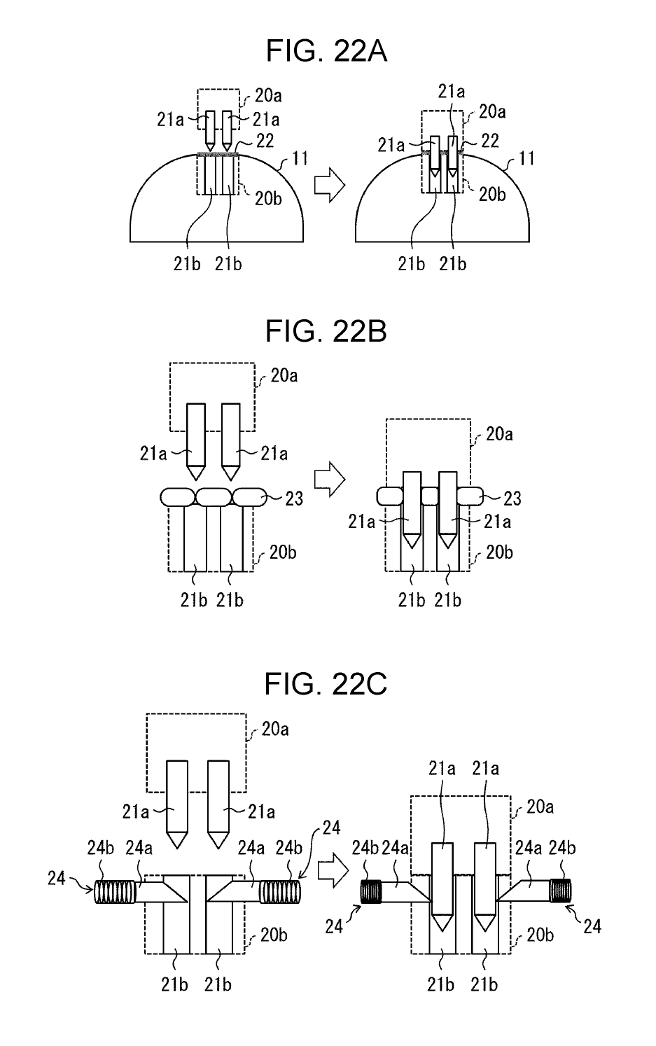

FIGS. 22A to 22C are schematic views illustrating a waterproof mechanism of a connection portion between the support rod side electrode 20a and the camera side electrode 20b. In an example illustrated in FIG. 22A, a waterproof film 22 (waterproof protective member) is disposed on a surface of the camera side electrode 20b of the camera 11. When the support rod side electrode 20a and the camera side electrode 20b are connected to each other, a terminal 21a (first terminal) on the support rod side electrode 20a side is inserted into a terminal 21b (second terminal) of the camera side electrode 20b. In this manner, the waterproof film 22 is broken. As a result, the waterproof film 22 is interposed between the support rod side electrode 20a and the camera side electrode 20b, except for the connection portion between the terminal 21a and the terminal 21b. Therefore, the connection portion between the support rod side electrode 20a and the camera side electrode 20b can be waterproofed. The waterproof film 22 is not particularly limited as long as the waterproof film 22 can be broken by the connection between the terminal 21a and the terminal 21b, and can be appropriately set in accordance with a configuration of the terminal 21a and the terminal 21b.

In an example illustrated in FIG. 22B, a perforated waterproof rubber 23 (perforated waterproof elastic member) is disposed on the surface of the camera side electrode 20b of the camera 11. A hole of the perforated waterproof rubber 23 is formed so as to correspond to the terminal 21a on the support rod side electrode 20a side. When the support rod side electrode 20a and the camera side electrode 20b are connected to each other, the terminal 21a on the support rod side electrode 20a side passes through the hole of the perforated waterproof rubber 23, and is inserted into the terminal 21b of the camera side electrode 20b. Therefore, the perforated waterproof rubber 23 is interposed between the support rod side electrode 20a and the camera side electrode 20b, except for the connection portion between the terminal 21a and the terminal 21b. Therefore, the connection portion between the support rod side electrode 20a and the camera side electrode 20b can be waterproofed.

In an example illustrated in FIG. 22C, a slide mechanism 24 is disposed in the camera side electrode 20b. The slide mechanism 24 holds a relative position of the terminal 21a with respect to the terminal 21b when the terminal 21a on the support rod side electrode 20a side is inserted into the terminal 21b of the camera side electrode 20b. The slide mechanism 24 includes two sliders 24a (slide members) disposed at positions for pinching the terminal 21b and two springs 24b (biasing members) which bias the two sliders 24a in a direction for pinching the terminal 21b.

The two sliders 24a are located so as to laterally intersect the terminal 21b by the biased two springs 24b. Two mutually facing end surfaces in the two sliders 24a are inclined surfaces which are inclined so that the gap between the two end surfaces decreases in an insertion direction of the terminal 21a. If the terminal 21a on the support rod side electrode 20a side is inserted into the terminal 21b of the camera side electrode 20b, the terminal 21a comes into contact with the above-described inclined surface of the slider 24a. Due to this contact, the two sliders 24a move so as to be apart from each other (in a direction opposite to the biasing direction of the spring 24b). In this manner, the terminal 21a is further moved, and the support rod side electrode 20a and the camera side electrode 20b are brought into close contact with each other so as to establish electrical connection therebetween. In this case, due to a biasing force of the spring 24b, the two sliders 24a press the terminal 21a in a direction for pinching the terminal 21a. Due to this pressing, the relative position of the terminal 21a is held with respect to the terminal 21b. As a result, a close contact state between the support rod side electrode 20a and the camera side electrode 20b is maintained, and the connection portion between the support rod side electrode 20a and the camera side electrode 20b can be waterproofed.

### Modification Example

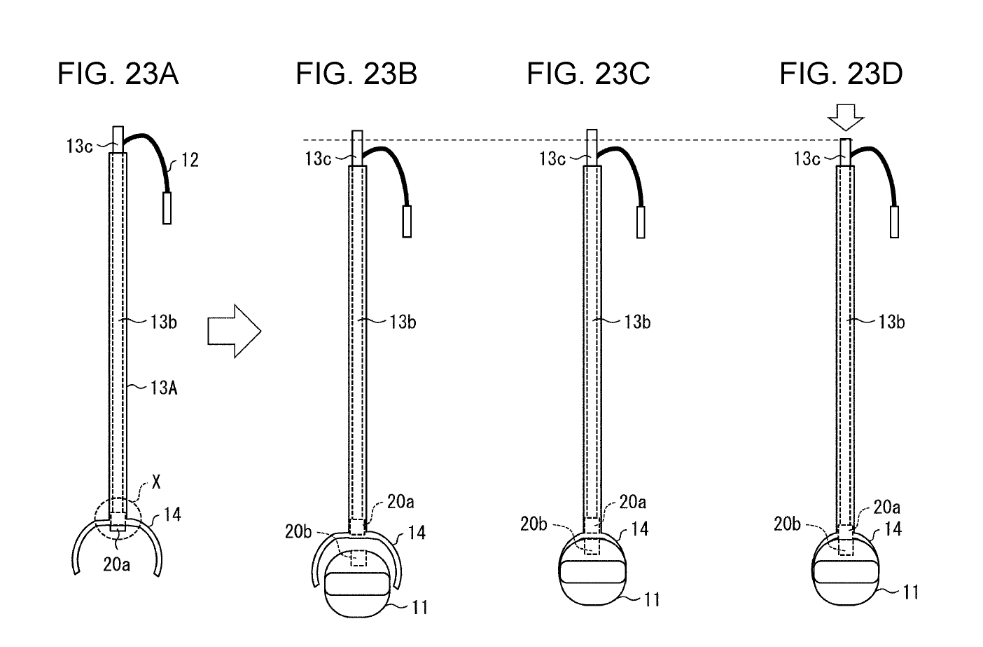

A modification example of the support rod 13A of the in-vivo imaging device according to the present embodiment will be described. FIGS. 23A to 23D are schematic views illustrating a configuration of the support rod 13A and the gripping portion 14 according to the modification example, and an operation method thereof.

As illustrated in FIGS. 23A to 23D, the support rod 13A includes an outer member 13b in a tubular shape, and an inner member 13c insertable into the outer member 13b. The inner member 13c extends to the gripping portion 14, and includes the support rod side electrode 20a in a distal end on the gripping portion 14 side. The support rod side electrode 20a is located in the connection portion X between the support rod 13A and the gripping portion 14, and is disposed so as to protrude from the gripping portion 14. The inner member 13c is pulled up with respect to the outer member 13b. In this manner, the support rod side electrode 20a is accommodated inside the outer member 13b without protruding from the gripping portion 14. The inner member 13c is pushed down with respect to the outer member 13b. In this manner, the support rod side electrode 20a protrudes from the gripping portion 14.

Since the support rod 13A has the configuration illustrated in FIG. 23A, the support rod side electrode 20a protrudes from the gripping portion 14 or is accommodated inside the outer member 13b in conjunction with the upward-downward movement of the inner member 13c with respect to the outer member 13b. Therefore, without depending on the timing to grip the camera 11 by using the gripping portion 14, it is possible to appropriately control the timing of the electrical connection between the support rod side electrode 20a and the camera side electrode 20b.

First, as illustrated in FIG. 23B, before the position of the camera 11 is held by causing the gripping portion 14 to grip the camera 11, the inner member 13c of the support rod 13A is pulled up. In this manner, the support rod side electrode 20a is accommodated inside the outer member 13b (before the camera is held). As illustrated in FIG. 23C, when the position of the camera 11 is held by causing the gripping portion 14 to grip the camera 11, a state where the inner member 13c of the support rod 13A is pulled up is maintained, and the support rod side electrode 20a remains accommodated inside the outer member 13b (when the camera is held). As illustrated in FIG. 23D, after the position of the camera 11 is held by causing the gripping portion 14 to grip the camera 11, the inner member 13c is pushed down with respect to the outer member 13b. In this manner, the support rod side electrode 20a protrudes from the gripping portion 14, and the support rod side electrode 20a and the camera side electrode 20b are electrically connected to each other.

According to the operation method using the support rod 13A of the modification example, when the position of the camera 11 is held by causing the gripping portion 14 to grip the camera 11 (step in FIG. 23C), the inner member 13c is pushed down with respect to the outer member 13b. In this manner, the support rod side electrode 20a and the camera side electrode 20b can be electrically connected to each other. In this way, according to the operation method using the support rod 13A of the modification example, without depending on the timing to grip the camera 11 by using the gripping portion 14, it is possible to appropriately control the timing of the electrical connection between the support rod side electrode 20a and the camera side electrode 20b.

### Embodiment 3

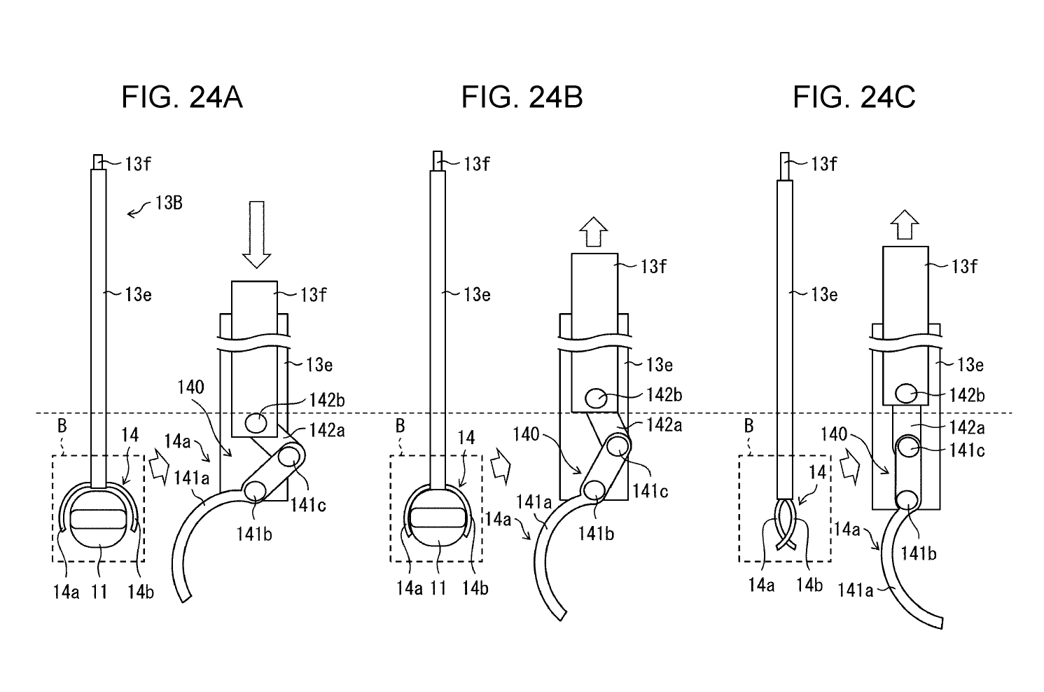

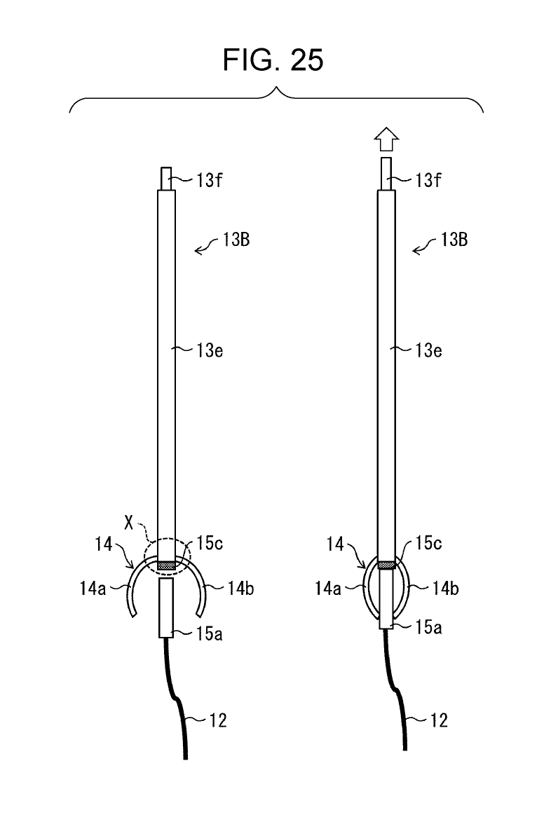

Referring to FIGS. 24A to 25C, a still further embodiment according to the disclosure will be described as follows. For the convenience of description, the same reference numerals will be given to members having functions which are the same as those of the members described in the above embodiment, and description thereof will be omitted.

FIGS. 24A to 24C are schematic views illustrating a configuration of a support rod 13B and the gripping portion 14 which are included in the in-vivo imaging device according to the present embodiment, and an operation method thereof. FIGS. 24A to 24C respectively illustrate enlarged sectional views of a portion B. In order to simplify the drawings, a configuration of the claw portion 14b is omitted in the enlarged sectional view of the portion B in each of FIGS. 24A to 24C. The configuration of the claw portion 14b is symmetrical to the configuration of the claw portion 14a illustrated in the enlarged sectional view of the portion B.

As illustrated in FIGS. 24A to 24C, the in-vivo imaging device according to the present embodiment is different from that according to Embodiment 1 and 2 as follows. The support rod 13B includes an outer member 13e in a tubular shape, and an inner member 13f insertable into the outer member 13e. The in-vivo imaging device according to the present embodiment includes a gripping mechanism 140 in which the gripping portion 14 grips the camera 11 in conjunction with the upward-downward movement of the inner member 13f.

The gripping mechanism 140 includes a claw member 141a, a link member 142a linking the claw member 141a and the inner member 13f with each other, and rotary shafts 141b, 141c, and 142b. A substantially central portion of the claw member 141a has the rotary shaft 141b. The rotary shaft 141b is fixed to the outer member 13e. Therefore, the claw member 141a is supported by the rotary shaft 141b rotatably with respect to the outer member 13e. An end portion of the claw member 141a on the link member 142a side has the rotary shaft 141c for being linked with the link member 142a. The link member 142a is supported by the rotary shaft 141c rotatably with respect to the claw member 141a. An end portion of the link member 142a on the inner member 13f side has the rotary shaft 142b for being linked with the inner member 13f. The rotary shaft 142b is fixed to the inner member 13f. Therefore, the link member 142a is supported by the rotary shaft 142b rotatably with respect to the inner member 13f.

As illustrated in FIG. 24A, when the inner member 13f is pushed down with respect to the outer member 13e, a distance decreases between the rotary shaft 141b and the rotary shaft 142b. In response to this decrease, an operation is performed by the rotary shaft 141c so that an angle decreases between the claw member 141a and the link member 142a. As a result, the claw member 141a is rotated around an axis of the rotary shaft 141b in a direction away from an axis of the outer member 13e. Although not illustrated, the claw member of the claw portion 14b is also rotated in the direction away from the axis of the outer member 13e by a similar mechanism. As a result, the gripping portion 14 is brought into an open state by the operation of pushing down the inner member 13f, and the distance increases between the claw portions 14a and 14b.

As illustrated in FIG. 24B, when the inner member 13f is pulled up with respect to the outer member 13e, the distance increases between the rotary shaft 141b and the rotary shaft 142b. In response to this increase, an operation is performed by the rotary shaft 141c so that the angle increases between the claw member 141a and the link member 142a. As a result, the claw member 141a is rotated around the axis of the rotary shaft 141b in a direction closer to the axis of the outer member 13e. Although not illustrated, the claw member of the claw portion 14b is also rotated in the direction closer to the axis of the outer member 13e by a similar mechanism. As a result, the gripping portion 14 is brought into a closed state by the operation of pulling up the inner member 13f, and the distance decreases between the claw portions 14a and 14b. In this manner, the gripping portion 14 can grip the camera 11.

As illustrated in FIG. 24C, when the inner member 13f is pulled up with respect to the outer member 13e without gripping the camera 11, the rotary shaft 141b, the rotary shaft 141c, and the rotary shaft 142b are aligned with each other on the same straight line. As a result, the claw member 141a is further rotated around the axis of the rotary shaft 141b in the direction closer to the axis of the outer member 13e. Therefore, the gripping portion 14 is brought into a state where the claw portion 14a and the claw portion 14b intersect each other.

In this way, the in-vivo imaging device according to the present embodiment includes the gripping mechanism 140 in which the gripping portion 14 grips the camera 11 in conjunction with the upward-downward movement of the inner member 13f. Therefore, it is possible to more appropriately set the timing to grip the camera 11 by using the gripping portion 14.

As illustrated in FIG. 25, the gripping portion 14 may be provided with the magnet 15c or the magnetic substance in the connection portion X connected to the support rod 13B. In this case, the magnet or the magnetic substance is attached to the distal end portion of the camera side cable connector 15a, and the magnet 15c magnetically attracts the camera side cable connector 15a. Since the magnet 15c is provided in this way, the support rod 13B having the gripping portion 14 can be used as the drawing tool for pulling up the camera side cable 12.

As illustrated in the left drawing in FIG. 25, in a state where the gripping portion 14 is open, the support rod 13B is located in the vicinity of the camera side cable connector 15a inside the body cavity. As illustrated in the right drawing in FIG. 25, the operation of pulling up the inner member 13f is performed. In conjunction with this operation, the gripping portion 14 is brought into a closed state, and the camera side cable 12 or the camera side cable connector 15a is gripped by the gripping portion 14, and is magnetically attracted to the magnet 15c. In that state, the camera side cable 12 is pulled outward of the body by pulling the support rod 13B outward of the body.

In this way, according to the configuration illustrated in FIG. 25, the camera side cable 12 or the camera side cable connector 15a can be gripped in conjunction with the operation of the inner member 13f. Therefore, it is possible to more appropriately set the timing to grip and pull the camera side cable 12 outward of the body by using the gripping portion 14.

### Summary

The in-vivo imaging device according to Aspect 1 of the disclosure includes the imaging unit (camera 11) that internally captures an image of the body cavity, the support member (support rod 13) insertable into the tubular instrument 31 to be introduced into the body, and the gripping portion 14 that is disposed at the in-vivo side end portion of the support member and grips the imaging unit. The gripping portion 14 is configured as follows. When the support member is pulled up outward of the body, the gripping portion 14 comes into contact with the in-vivo side end portion (in-vivo side end portion 31a) of the tubular instrument 31. In this manner, the gripping portion 14 is deformed so as to grip the imaging unit.

According to the above-described configuration, when the support member is pulled up outward of the body, the gripping portion comes into contact with the in-vivo side end portion of the tubular instrument. In this manner, the gripping portion is deformed so as to grip the imaging unit. Therefore, when carrying out installation work of the imaging unit inside the body, the operator locates the gripping portion in the vicinity of the imaging unit by pushing down the support rod through the tubular instrument. Thereafter, the operator pulls up the support rod so as to bring the gripping portion into contact with the in-vivo side end portion of the tubular instrument. In this manner, the position of the imaging unit can be held. In this way, the position of the imaging unit can be held only by performing the operation of bringing the gripping portion into contact with the in-vivo side end portion of the tubular instrument by pulling up the support rod. Accordingly, the installation work of the imaging unit can be simplified. Even after the installation, the angle and position of the imaging unit can be finely adjusted.

Therefore, according to the above-described configuration, it is possible to realize the in-vivo imaging device which can simplify the installation work of the camera inside the body, and which can finely adjust the angle and position of the camera even after the installation.

In the in-vivo imaging device according to Aspect 2 of the disclosure, in Aspect 1 described above, the gripping portion 14 may be configured to have at least one claw portion 14a which comes into contact with the in-vivo side end portion 31a of the tubular instrument 31.

In the in-vivo imaging device according to Aspect 3 of the disclosure, in Aspect 1 described above, the gripping portion 14 may be configured to have the annular portion 14k which surrounds the imaging unit (camera 11) and comes into contact with the in-vivo side end portion 31a of the tubular instrument 31.

In the in-vivo imaging device according to Aspect 4 of the disclosure, in Aspect 1 described above, the imaging unit (camera 11) has the pickup portion 11c which is smaller than the imaging unit. The gripping portion 14 is configured to pick up the pickup portion 11c.

According to the above-described configuration, the gripping portion 14 is configured to pick up the pickup portion 11c which is smaller than the imaging unit. Accordingly, the gripping portion 14 can be small in size.

In the in-vivo imaging device according to Aspect 5 of the disclosure, in Aspects 1 to 4 described above, it is preferable to adopt a configuration as follows. The in-vivo imaging device includes the cable (camera side cable 12) connectable to the imaging unit (camera 11). The cable is disposed inside the support member (support rod 13). The gripping portion 14 has at least one support member side electrode (support rod side electrode 20a) for electrically connecting the imaging unit and the cable to each other.

According to the above-described configuration, the support member side electrode for electrically connecting the imaging unit and the cable to each other is disposed in the gripping portion 14. Therefore, when the position of the imaging unit located inside the body is held, the gripping portion can grip the imaging unit, and can electrically connect the cable and the imaging unit to each other. Accordingly, the installation work of the camera inside the body can be simplified.

In the in-vivo imaging device according to Aspect 6 of the disclosure, in Aspect 5 described above, a configuration may be adopted in which the support member side electrode (support rod side electrode 20a) is disposed in the connection portion X between the support member (support rod 13) and the gripping portion 14.

In the in-vivo imaging device according to Aspect 7 of the disclosure, in Aspect 5 described above, a configuration may be adopted in which at least the one support member side electrode (support rod side electrode 20a) includes two or more support member side electrodes in the gripping portion 14, and the two or more support member side electrodes are located so as to be separated from each other.

In this manner, short-circuit between the electrodes can be avoided.

In the in-vivo imaging device according to Aspect 8 of the disclosure, in Aspects 5 to 7 described above, a configuration may be adopted as follows. The imaging unit side electrode (camera side electrode 20b) connectable to the support member side electrode (support rod side electrode 20a) is disposed in the imaging unit (camera 11). The imaging unit side electrode is located at the position where the imaging unit side electrode comes into contact with the support member side electrode when the imaging unit is gripped by the gripping portion 14.

In this manner, the cable and the imaging unit are electrically connected to each other with the more simplified configuration.

In the in-vivo imaging device according to Aspect 9 of the disclosure, in Aspect 8 described above, a configuration may be adopted as follows. The waterproof protective member (waterproof film 22) is disposed between the support member side electrode (support rod side electrode 20a) and the imaging unit side electrode (camera side electrode 20b). A terminal 21a of the support member side electrode or a terminal of the imaging unit side electrode penetrates the waterproof protective member.

In this manner, the connection portion between the support member side electrode and the imaging unit side electrode can be waterproofed.

In the in-vivo imaging device according to Aspect 10 of the disclosure, in Aspect 8 described above, a configuration may be adopted as follows. The perforated waterproof elastic member (perforated waterproof rubber 23) is disposed between the support member side electrode (support rod side electrode 20a) and the imaging unit side electrode (camera side electrode 20b). A terminal 21a of the support member side electrode or a terminal of the imaging unit side electrode passes through the hole of the waterproof elastic member.

In the in-vivo imaging device according to Aspect 11 of the disclosure, in Aspect 8 described above, a configuration may be adopted as follows. The in-vivo imaging device further includes the slide mechanism 24 that holds the relative position of the first terminal (terminal 21a) of the support member side electrode (support rod side electrode 20a) with respect to a second terminal (terminal 21b) of the imaging unit side electrode (camera side electrode 20b), when the first terminal is inserted into the second terminal. The slide mechanism 24 includes at least two slide members (sliders 24a) disposed at the position where the two slide members hold the first terminal inserted into the second terminal, and the biasing member (spring 24b) which biases the slide members in the direction in which the first terminal is held.

According to the above-described configuration, when the first terminal on the support member side electrode is inserted into the second terminal of the imaging unit side electrode, the slide mechanism 24 holds the relative position of the first terminal with respect to the second terminal. Accordingly, the support member side electrode and the imaging unit side electrode are maintained in the close contact state. Therefore, the connection portion between the support member side electrode and the imaging unit side electrode can be waterproofed.

In the in-vivo imaging device according to Aspect 12 of the disclosure, in Aspects 5 to 11 described above, a configuration may be adopted as follows. The support member includes the outer member 13b in a tubular shape and the inner member 13c insertable into the outer member 13b. The inner member 13c extends up to the gripping portion 14, and the support member side electrode (support rod side electrode 20a) is disposed at the distal end on the gripping portion 14 side of the inner member. The support member side electrode protrudes from the gripping portion 14 or is accommodated inside the outer member 13b in conjunction with the upward-downward movement of the inner member 13c with respect to the outer member 13b.

According to the above-described configuration, the support rod side electrode protrudes from the gripping portion 14 or is accommodated inside the outer member 13b in conjunction with the upward-downward movement of the inner member 13c with respect to the outer member 13b. Therefore, irrespective of the timing of gripping the imaging unit by using the gripping portion 14, the timing of electrically connecting the support member side electrode and the imaging unit side electrode to each other can be appropriately controlled.

In the in-vivo imaging device according to Aspect 13 of the disclosure, in Aspects 1 to 12 described above, a configuration may be adopted in which the groove portion (groove 11a) appropriate for the shape of the gripping portion 14 is formed in the imaging unit (camera 11).

In this manner, the imaging unit can be more stably gripped by the gripping portion 14.

The in-vivo imaging device according to Aspect 14 of the disclosure is configured as follows. The in-vivo imaging device includes the imaging unit (camera 11) that internally captures an image of the body cavity, the support member (support rod 13) insertable into the tubular instrument 31 introduced into the body, and the gripping portion 14 that is disposed at the in-vivo side end portion of the support member and grips the imaging unit. The gripping portion 14 has the sucker portion 14l which suctions the imaging unit.

According to the above-described configuration, the gripping portion has the sucker portion 14l which suctions the imaging unit. Therefore, when carrying out installation work of the imaging unit inside the body, the operator locates the gripping portion in the vicinity of the imaging unit by pushing down the support rod through the tubular instrument. Thereafter, the operator can hold the position of the imaging unit by causing the sucker portion 14l to suction the imaging unit. In this way, the position of the imaging unit can be held only by performing the operation of causing the sucker portion 14l to suction the imaging unit. Accordingly, the installation work of the imaging unit can be simplified. Even after the installation, the angle and position of the imaging unit can be finely adjusted.

Therefore, according to the above-described configuration, it is possible to realize the in-vivo imaging device which can simplify the installation work of the camera inside the body, and which can finely adjust the angle and position of the camera even after the installation.

In the in-vivo imaging device according to Aspect 15 of the disclosure, in Aspect 14 described above, a configuration may be adopted in which the recess portion 11b appropriate for the shape of the sucker portion 14l is formed in the imaging unit (camera 11).

In this manner, the imaging unit can be more stably gripped by the sucker portion 14l.

The in-vivo imaging device according to Aspect 16 of the disclosure is configured as follow. The in-vivo imaging device includes the imaging unit (camera 11) that internally captures an image of the body cavity, the support member (support rod 13) insertable into the tubular instrument 31 introduced into the body, and the gripping portion 14 that is disposed at the in-vivo side end portion (in-vivo side end portion 31a) of the support member and grips the imaging unit. The support member includes the outer member 13e in a tubular shape, the inner member 13f insertable into the outer member 13e, and the gripping mechanism 140 with which the gripping portion 14 grips the imaging unit in conjunction with the upward-downward movement of the inner member 13f. The gripping mechanism 140 includes the claw member 141a which is supported rotatably with respect to the outer member 13e, and the link member 142a which links the claw member 141a and the inner member 13f with each other and which is supported rotatably with respect to the claw member 141a. The link member 142a is supported rotatably with respect to the inner member 13f.

According to the above-described configuration, it is possible to realize the in-vivo imaging device which can simplify the installation work of the camera inside the body, and which can finely adjust the angle and position of the camera even after the installation.

According to the above-described configuration, the in-vivo imaging device includes the gripping mechanism 140 in which the gripping portion 14 grips the camera 11 in conjunction with the upward-downward movement of the inner member 13f. Therefore, it is possible to more appropriately set the timing of gripping the camera 11 by using the gripping portion 14.

In the in-vivo imaging device according to Aspect 17 of the disclosure, in Aspects 1 to 16 described above, it is preferable to adopt a configuration as follows. The in-vivo imaging device further includes the cable (camera side cable) connectable to the imaging unit (camera 11). The gripping portion 14 has the magnet 15c or the magnetic substance which magnetically attracts the external instrument connecting connector (camera side cable connector 15a) in the cable at the connection portion X between the gripping portion 14 and the support member (support rod 13).

According to the above-described configuration, the gripping portion 14 has the magnet 15c or the magnetic substance which magnetically attracts the external instrument connecting connector in the cable at the connection portion X between the gripping portion 14 and the support member. Therefore, the support member having the gripping portion 14 can be used as the drawing tool for pulling up the above-described cable.

In the in-vivo imaging device according to Aspect 18 of the disclosure, in Aspects 1 to 17 described above, it is preferable to adopt a configuration as follows. The imaging unit (camera 11) has the shape extending in the longitudinal direction. The connection portion X between the support member (support rod 13) and the gripping portion 14 is configured to include the elastic material. The gripping portion 14 has the curved shape which is curved so as to correspond to the outer peripheral shape of the imaging unit, is more rigid than the connection portion X, and is open wider than the width of the imaging unit in the short direction.

The in-vivo monitoring camera system according to Aspect 19 of the disclosure, may include the in-vivo imaging device according to any one of Aspects 1 to 18 described above, the cable (camera side cable) connectable to the imaging unit (camera 11), and the control system that is located outside the body, that is electrically connectable to the cable, and that includes at least the display device (display 18).

According to the above-described configuration, the control system includes the display device. Therefore, an image of the body internally captured can be displayed.

The disclosure is not limited to the above-described embodiments, and can be modified in various ways within the scope of the aspects of the disclosure. Any embodiment obtained by appropriately combining technical ideas respectively described in the mutually different embodiments with each other may be included in the technical scope of the disclosure. Furthermore, and new technical features can be formed by combining the technical ideas respectively described in each of the embodiments.

The present disclosure contains subject matter related to that disclosed in Japanese Priority Patent Application JP 2017-167930 filed in the Japan Patent Office on Aug. 31, 2017, the entire contents of which are hereby incorporated by reference.

It should be understood by those skilled in the art that various modifications, combinations, sub-combinations and alterations may occur depending on design requirements and other factors insofar as they are within the scope of the appended claims or the equivalents thereof.

What is claimed is:

1. An in-vivo imaging device comprising:
an imaging unit that internally captures an image of a body cavity;
a support member insertable into a tubular instrument to be introduced into a body; and
a gripping portion that is disposed at an in-vivo side end portion of the support member and grips the imaging unit, wherein
when the support member is pulled up outward of the body, the gripping portion is deformed by coming into contact with the in-vivo side end portion of the tubular instrument so as to grip the imaging unit.

2. The in-vivo imaging device according to claim 1, wherein
the gripping portion has at least one claw portion which comes into contact with the in-vivo side end portion of the tubular instrument.

3. The in-vivo imaging device according to claim 1, wherein
the gripping portion has an annular portion which surrounds the imaging unit and comes into contact with the in-vivo side end portion of the tubular instrument.

4. The in-vivo imaging device according to claim 1, wherein
the imaging unit has a pickup portion which is smaller than the imaging unit, and
the gripping portion is configured to pick up the pickup portion.

5. The in-vivo imaging device according to claim 1, further comprising:
a cable connectable to the imaging unit, wherein
the cable is disposed inside the support member, and
the gripping portion has at least one support member side electrode for electrically connecting the imaging unit and the cable to each other.

6. The in-vivo imaging device according to claim 5, wherein
At least the one support member side electrode is disposed in a connection portion between the support member and the gripping portion.

7. The in-vivo imaging device according to claim 5, wherein
at least the one support member side electrode is disposed in the two or more gripping portions, the two or more gripping portions being located so as to be separated from each other.

8. The in-vivo imaging device according to claim 5, wherein
the imaging unit has an imaging unit side electrode connectable to at least the one support member side electrode, and
the imaging unit side electrode is located at a position where the imaging unit side electrode comes into contact with at least the one support member side electrode when the imaging unit is gripped by the gripping portion.

9. The in-vivo imaging device according to claim 8, wherein
a waterproof protective member is disposed between at least the one support member side electrode and the imaging unit side electrode, and
a terminal of at least the one support member side electrode or a terminal of the imaging unit side electrode penetrates the waterproof protective member.

10. The in-vivo imaging device according to claim 8, wherein
a perforated waterproof elastic member is disposed between at least the one support member side electrode and the imaging unit side electrode, and
a terminal of at least the one support member side electrode or a terminal of the imaging unit side electrode passes through a hole of the waterproof elastic member.

11. The in-vivo imaging device according to claim 8, further comprising:
a slide mechanism that holds a relative position of a first terminal of at least the one support member side electrode with respect to a second terminal of the imaging unit side electrode, when the first terminal is inserted into the second terminal, wherein
the slide mechanism includes at least two slide members disposed at a position where the at least two slide members hold the first terminal inserted into the second terminal, and a biasing member which biases the at least two slide members in a direction in which the first terminal is held.

12. The in-vivo imaging device according to claim 5, wherein
the support member includes an outer member in a tubular shape and an inner member insertable into the outer member,
the inner member extends up to the gripping portion, and at least the one support member side electrode is disposed at a distal end on the gripping portion side of the inner member, and
at least the one support member side electrode protrudes from the gripping portion or is accommodated inside the outer member in conjunction with an upward-downward movement of the inner member with respect to the outer member.

13. The in-vivo imaging device according to claim 1, wherein
the imaging unit has a groove portion appropriate for a shape of the gripping portion.

14. An in-vivo imaging device comprising:
an imaging unit that internally captures an image of a body cavity;
a support member insertable into a tubular instrument to be introduced into a body; and
a gripping portion that is disposed at an in-vivo side end portion of the support member and grips the imaging unit, wherein
the gripping portion has a sucker portion which suctions the imaging unit.

15. The in-vivo imaging device according to claim 14, wherein
the imaging unit has a recess portion appropriate for a shape of the sucker portion.

16. An in-vivo imaging device comprising:
an imaging unit that internally captures an image of a body cavity;
a support member insertable into a tubular instrument to be introduced into a body; and
a gripping portion that is disposed at an in-vivo side end portion of the support member and grips the imaging unit, wherein
the support member includes an outer member in a tubular shape, an inner member insertable into the outer member, and a gripping mechanism with which the gripping portion grips the imaging unit in conjunction with an upward-downward movement of the inner member,
the gripping mechanism includes a claw member which is supported rotatably with respect to the outer member, and a link member which links the claw member and the inner member with each other and which is supported rotatably with respect to the claw member, and
the link member is supported rotatably with respect to the inner member.

17. The in-vivo imaging device according to claim 1, further comprising:
a cable connectable to the imaging unit, wherein
the gripping portion has a magnet or a magnetic substance which magnetically attracts an external instrument connecting connector in the cable at a connection portion between the gripping portion and the support member.

18. The in-vivo imaging device according to claim 1, wherein
the imaging unit has a shape extending in a longitudinal direction,
a connection portion between the support member and the gripping portion is formed of an elastic material, and
the gripping portion has a curved shape which is curved so as to correspond to an outer peripheral shape of the imaging unit, is more rigid than the connection portion, and is open wider than a width of the imaging unit in a short direction.

19. An in-vivo monitoring camera system comprising:
the in-vivo imaging device according to claim 1;
a cable connectable to the imaging unit; and
a control system that is located outside a body, that is electrically connectable to the cable, and that includes at least a display device.
